(19) United States
(12) Patent Application Publication
(10) Pub. No.: US 2018/0072988 A1
(43) Pub. Date: Mar. 15, 2018

(54) GENERATION OF FUNCTIONAL CELLS FROM STEM CELLS

(71) Applicant: AGENCY FOR SCIENCE, TECHNOLOGY AND RESEARCH, Singapore (SG)

(72) Inventors: Xuyang SUN, Singapore (SG); Bing LIM, Singapore (SG)

(21) Appl. No.: 15/565,662

(22) PCT Filed: Apr. 11, 2016
(86) PCT No.: PCT/SG2016/050176

§ 371 Date: Oct. 10, 2017

(30) Foreign Application Priority Data
Apr. 10, 2015 (SG) 10201502869T

(51) Int. Cl. C12N 5/0793; G01N 33/50; G01N 33/569; C12N 15/85

(52) U.S. Cl. CPC C12N 5/0619; G01N 33/5073; G01N 33/56966; C12N 15/85; G01N 2800/28; C12N 2501/65; C12N 2502/086; C12N 2506/02; C12N 2501/60

(57) ABSTRACT

The present disclosure provides a method of directly converting a stem cell into a lineage specific cell, comprising the steps of a) transfecting a stem cell with at least one expression vector comprising i) one or more cell lineage reprogramming factors operably linked to an inducible promoter and ii) a selection marker; and b) inducing the transfected stem cell from stem a) with an inducing agent to directly convert said stem cell into a lineage-specific cell. Particularly exemplified are methods of transfecting a stem cell with SA-ASCL1 (phospho-mutant), DLX2, LHX6 and miR-9/9*-124 linked to a doxycycline inducible promoter to convert the stem cell into an inhibitory neuron and transfecting with NeuroD2 linked to a doxycycline inducible promoter to convert a stem cell into an excitatory neuron. Methods of screening one or more factors and/or one or more genetic mutations that modulate a pre-selected activity of the lineage specific cell, kits and directly convertible stem cells obtained using method of the invention are also provided.

### CROSS-REFERENCE TO RELATED APPLICATIONS

This application claims the benefit of priority of Singapore application No. 10201502869T, filed 10 Apr. 2015, the contents of it being hereby incorporated by reference in its entirety for all purposes.

### FIELD OF THE INVENTION

The present invention relates generally to the field of biotechnology. In particular, the present invention relates to methods for differentiating a pluripotent or multipotent stem cell into multiple cell lineages. The present invention further relates to culture mediums and kits for use in performing the methods as described herein.

### BACKGROUND OF THE INVENTION

At multiple developmental junctures, lineage-specifying transcription factors (TFs) direct multipotent progenitors towards a single lineage outcome and repress alternate fates, ensuring a unilateral lineage decision.

The ability to differentiate stem cells such as human pluripotent stem cells (hPSC) into committed cell-types holds great benefit for cell replacement therapy, drug screening and discovery, mechanistic studies of dysfunction and other downstream applications.

An example of a cell lineage that stem cells can differentiate to is the neuronal lineage. Various methods to differentiate stem cells into cells of the neuronal lineage are known. These methods mimic the developmental signaling that occurs during biogenesis of neurons to generate neural progenitors and subsequently differentiate these progenitors into functional neurons. However, these methods typically involve multiple intermediate stages that require varying combinations of recombinant growth factors and small molecules, and eventually yield mixtures of both non-neuronal and neuronal cells with variable functional properties. The protracted timeline required to attain neuronal maturity and synaptic competence is a further limitation because this process can take as long as 30 weeks.

Accordingly, there is a need for a method to obtain functional lineage specific cells from stem cells that overcomes, or at least ameliorates, one or more of the disadvantages stated above.

### SUMMARY

In one aspect, there is provided a method of directly converting a stem cell into a lineage specific cell comprising:
a) transfecting said stem cell with at least one expression vector comprising i) one or more cell lineage reprogramming factors operably linked to an inducible promoter and ii) a selection marker; and
b) inducing said transfected stem cell from step a) with an inducing agent to directly convert said stem cell into at least one lineage specific cell.

In another aspect, there is provided a method of generating a directly convertible stem cell, said method comprising the steps of:
a) transfecting a stem cell with an expression vector comprising i) one or more cell lineage reprogramming factors operably linked to an inducible promoter and ii) a selection marker operably linked to a constitutive promoter; and
b) screening the transfected stem cell for expression of the selection marker to generate said directly convertible stem cell,
   wherein said directly convertible stem cell is capable of direct conversion into an induced lineage specific cell.

In one aspect, there is provided a directly convertible stem cell comprising i) one or more reprogramming factors operably linked to an inducible promoter and ii) a selection marker operably linked to a constitutive promoter, wherein said directly convertible stem cell is capable of direct conversion into an induced lineage specific cell.

In one aspect, there is provided a method of screening one or more factors and/or one or more genetic mutations that modulate a pre-selected activity of the induced lineage specific cell according to any one of the preceding claims, comprising the steps of:
a) culturing said induced lineage specific cell in the presence of one or more factors and/or one or more genetic mutations;
b) measuring the pre-selected activity of the lineage specific cell of step a); and
c) comparing the measurement of b) relative to the measurement of the pre-selected activity in the lineage specific cell that has not been cultured in the presence of the said one or more factors or genetic mutations,

wherein the difference in the measurement of the pre-selected activity of the lineage specific cell in c) indicates that the one or more factors or genetic mutations modulates the pre-selected activity of the lineage specific cell.

In one aspect, there is provided a kit for generating an induced lineage specific cell, comprising,
a) a directly convertible stem cell as described herein;
b) an inducer; and optionally instructions for use.

In one aspect, there is provided a method of directly converting a stem cell into a lineage specific cell comprising:
transfecting said stem cell with at least one expression vector comprising i) one or more cell lineage reprogramming factors operably linked to a constitutive promoter.

In one aspect, there is provided a method of directly converting a stem cell into a lineage specific cell comprising:
transfecting said stem cell with at least one expression vector comprising i) one or more cell lineage reprogramming factors operably linked to an inducible promoter.

In one aspect, there is provided a stem cell directly convertible into an inhibitory neuron comprising:
i) SA-ASCL1, DLX2, LHX6 and miR-9/9*-124 linked to a doxycycline inducible promoter; and
ii) a selection marker operably linked to a constitutive promoter.

In one aspect, there is provided a stem cell directly convertible into an excitatory neuron comprising:
i) NeuroD2 linked to a doxycycline inducible promoter; and
ii) a selection marker operably linked to a constitutive promoter.

In another aspect, there is provided a method of screening an agent using a cell obtained by the method disclosed herein comprising:
i) contacting said cell with the agent;
ii) measuring a pre-selected activity of the agent on the cell and comparing this to a cell that has not been contacted with the agent; and
iii) detecting the activity of the agent on said cell.

### DEFINITIONS

As used herein, the term “stem cells” include but are not limited to undifferentiated cells defined by their ability at the single cell level to both self-renew and differentiate to produce progeny cells, including self-renewing progenitors, non-renewing progenitors, and terminally differentiated cells. For example, “stem cells” may include (1) totipotent stem cells; (2) pluripotent stem cells; (3) multipotent stem cells; (4) oligopotent stem cells; and (5) unipotent stem cells.

As used herein, the term “totipotency” refers to a cell with a developmental potential to make all of the cells in the adult body as well as the extra-embryonic tissues, including the placenta. The fertilized egg (zygote) is totipotent, as are the cells (blastomeres) of the morula (up to the 16-cell stage following fertilization).

As used herein, the term “pluripotent stem cell” (PSC) refers to a cell with the developmental potential, under different conditions, to differentiate to cell types characteristic of all three germ cell layers, i.e., endodeini (e.g., gut tissue), mesoderm (including blood, muscle, and vessels), and ectoderm (such as skin and nerve). The developmental competency of a cell to differentiate to all three germ layers can be determined using, for example, a nude mouse teratoma formation assay. In some embodiments, pluripotency can also be evidenced by the expression of embryonic stem (ES) cell markers, although the preferred test for pluripotency of a cell or population of cells generated using the compositions and methods described herein is the demonstration that a cell has the developmental potential to differentiate into cells of each of the three germ layers.

As used herein, the term “induced pluripotent stem cells” or, iPSCs, means that the stem cells are produced from differentiated adult cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “embryonic stem cell” refers to naturally occurring pluripotent stem cells of the inner cell mass of the embryonic blastocyst. Such cells can similarly be obtained from the inner cell mass of blastocysts derived from somatic cell nuclear transfer. Embryonic stem cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta, i.e., are not totipotent.

As used herein, the term “multipotent stem cell” refers to a cell that has the developmental potential to differentiate into cells of one or more germ layers, but not all three. Thus, a multipotent cell can also be termed a “partially differentiated cell.” Multipotent cells are well known in the art, and examples of multipotent cells include adult stem cells, such as for example, hematopoietic stern cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

As used herein, the term “differentiation” is the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein, the term “undifferentiated cell” refers to a cell in an undifferentiated state that has the property of self-renewal and has the developmental potential to differentiate into multiple cell types, without a specific implied meaning regarding developmental potential (i.e., totipotent, pluripotent, multipotent, etc.).

As used herein, the term “progenitor cell” refers to cells that have greater developmental potential, i.e., a cellular phenotype that is more primitive (e.g., is at an earlier step along a developmental pathway or progression) relative to a cell which it can give rise to by differentiation. Often, progenitor cells have significant or very high proliferative potential. Progenitor cells can give rise to multiple distinct cells having lower developmental potential, i.e., differentiated cell types, or to a single differentiated cell type, depending on the developmental pathway and on the environment in which the cells develop and differentiate.

As used herein, the term “reprogramming” in the context of a cell or cell lineage refers to the conversion of a specific cell type to another cell type. Accordingly, “reprogramming factor” refers to a molecule that is capable of reprogramming a specific cell type to another cell type.

As used herein, the term “efficiency” in the context of reprogramming means that conversion of a specific cell type to another cell type occurs at a frequency of at least about 50%. In other words, reprogramming efficiency of at least about 50% means that at least about 50% of cells of a specific cell type is converted to another cell type.

As used herein, the tenn “markers” refers to nucleic acid or polypeptide molecule that is differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

As used herein, the term “inducible” in the context of a promoter refers to a promoter whose activity may be stimulated by an agent. Presence of the agent stimulates promoter activity which in turn drives expression of the gene that is under the control of the inducible promoter. In the absence of the agent, the promoter is inactive and the gene that is under the control of the inducible promoter is not expressed.

As used herein, the term “constitutive” in the context of a promoter refers to a promoter which is consistently active. A gene that is under the control of a constitutive promoter is continually expressed.

As used herein., the term “transcription factor” refers to proteins that bind to DNA and regulate transcription. Transcription factors may comprise DNA-binding domains which recognise and bind to specific sequences of DNA to regulate transcription. Transcription factors commonly recognise and bind to promoter and/or enhancer regions and may activate or repress gene expression.

As used herein, “microRNAs” or a “microRNA” molecule refers to a short, non-coding RNA which can negatively regulate expression of one or more genes at post-transcriptional level.

As used herein the phrase “culture medium” refers to a liquid substance used to support the growth of stem cells and any of the cell lineages. The culture medium used by the invention according to some embodiments can be a liquid-based medium, for example water, which may comprise a combination of substances such as salts, nutrients, minerals, vitamins, amino acids, nucleic acids, proteins such as cytokines, growth factors and hormones.

As used herein, the term “feeder cell” refers to feeder cells (e.g., fibroblasts) that maintain stem cells in a proliferative state when the stem cells are co-cultured on the feeder cells or when the pluripotent stem cells are cultured on a matrix (e.g., an extracellular matrix, a synthetic matrix) in the presence of a conditioned medium generated by the feeder cells. The support of the feeder cells depends on the structure of the feeder cells while in culture (e.g., the three dimensional matrix formed by culturing the feeder cells in a tissue culture plate), function of the feeder cells (e.g., the secretion of growth factors, nutrients and hormones by the feeder cells, the growth rate of the feeder cells, the expansion ability of the feeder cells before senescence) and/or the attachment of the stem cells to the feeder cell layer(s).

As used herein, the term “cortical network” refers to a group of neurons that are interconnected via one or more synapses. A cortical network may comprise of a group of neurons in vitro or ex vivo. An in vitro or ex vivo cortical network mimics the human cortex in the type of neurons present.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DISCLOSURE OF OPTIONAL EMBODIMENTS

Before the present inventions are described, it is to be understood that this invention is not limited to particular embodiments described, as such may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

In one embodiment, there is provided a method of directly converting a stem cell into a lineage specific cell. In particular, the method comprises: transfecting said stem cell with at least one expression vector comprising i) one or more cell lineage reprogramming factors operably linked to an inducible promoter and ii) a selection marker; and inducing said transfected stem cells from step a) with an inducing agent to directly convert said stem cell into a lineage specific cell.

In one embodiment, the at least one expression vector may comprise a selection marker operably linked to a constitutive promoter.

The method as described herein may further comprise the step of selecting the transfected stem cell for expression of the selection marker, prior to inducing the cells.

In some embodiments, the selection marker may be an antibiotic resistance gene selected from the group consisting of puromycin, blasticidin, hygromycin, zeocin and neomycin.

In a preferred embodiment, the antibiotic resistance gene is blasticidin and/or hygromycin.

The constitutive promoter may be selected from the group consisting of phosphoglycerate kinase (PGK), elongation factor 1-α(EF1α), ubiquitin C (UBC), (β-actin and cytomegalovirus (CMV) enhancer/chicken β-actin promoter (CAG).

In a preferred embodiment, the constitutive promoter is PGK or EF1α.

In some embodiments, the method may further comprise the step of transfecting the stem cell with an expression vector comprising a transactivator capable of inducing the inducible promoter in the presence of an inducer. The inducing agent may be selected from the group consisting of doxycycline and cumate. It will be understood that when the inducing agent is doxycycline, the transactivator is a reverse tetracycline-controlled transactivator (rtTA). It will also be understood that when the inducing agent is cumate, the transactivator is a reverse cumate activator (rcTA).

The expression vector may be an integrating or non-integrating vector. In some embodiments, the integrating vector may be a retroviral or lentiviral expression vector. In some embodiments, the non-integrating vector is a sendai virus, adeno-associated virus (AAV) or episomal DNA. In a preferred embodiment, the vector is a lentiviral expression vector.

The method as described herein may further comprise the step of enriching the selected cells using one or more selection steps. In some embodiments, the selection step may be selected from the group consisting of antibiotic selection, fluorescence activated cell sorting (FACS), magnetic activated cell sorting (MACS), or single clone isolation and expansion.

The stem cell may be an embryonic stem cell (ESC) or induced pluripotent stem cell (iPSC). In some embodiments, the stem cell may be a primate or non-primate stem cell. Where a primate stem cell is selected, the stem cell may be a human stem cell. In a preferred embodiment, the embryonic stem cell is a human embryonic stem cell.

In some embodiments, the stem cell may be a stem cell line. The stem cell line may be cultured as a two-dimensional cell culture or a three-dimensional cell culture.

It is an advantage of the method as described herein that the lineage specific cell is generated at an efficiency of at least about 50%, at least about 60%, at least about 70%, at least about 80%, at least about 90%, at least about 99% and about 100%. In a preferred embodiment, the efficiency is at least about 70%. In yet another preferred embodiment, the efficiency is at least about 99%.

The lineage specific cell may be a population of cells cultured as a two-dimensional cell culture or a three-dimensional cell culture. In some embodiments, the lineage specific cell may be a cell of the ectoderm, mesoderm or endoderm lineage. In a preferred embodiment, the cell of the ectoderm lineage may be a neural cell. The neural cell may be selected from the group consisting of excitatory neurons, inhibitory neurons, dopamine neurons, serotonin neurons, medium spiny neurons, basal forebrain cholinergic neuron, oligodendrocytes, astrocytes and motor neurons. In a further preferred embodiment, the neural cell may be an excitatory neuron or an inhibitory neuron. In another embodiment, the neural cell is at least one cell of a cortical network. A minimal cortical network comprises an excitatory and an inhibitory neuron. It will be understood that the ratio of types of neurons within a cortical network may vary. For example, a cortical network may comprise at least about 70% excitatory neurons and at least about 30% inhibitory neurons, at least about 75% excitatory neurons and at least about 25% inhibitory neurons, at least about 80% excitatory neurons and at least about 20% inhibitory neurons, at least about 85% excitatory neurons and at least about 15% inhibitory neurons and at least about 90% excitatory neurons and at least about 10% inhibitory neurons. In a preferred embodiment, a cortical network may comprise at least about 75% excitatory and at least about 25% inhibitory neurons. In yet another preferred embodiment, a cortical network may comprise at least about 80% excitatory and at least about 20% inhibitory neurons.

In one embodiment, the inhibitory neuron may be selected from the group consisting of parvalbumin (PV) type, somatostatin (SOM) type, calbindin (CB) type, calretinin (Cr) type, vasoactive intestinal polypeptide (VIP) type, Reelin type, neuropeptide Y (NPY) type, neuronal nitric oxide synthase (nNOS) type and 5HT3aR expressing neurons. In a preferred embodiment, the inhibitory neuron is a SOM type, CR type, CB type or NPY type neuron.

In some embodiments, the cell of the mesoderm lineage may be a cardiac cell. The cardiac cell may be selected from the group consisting of cardiomyocytes, endothelial cells, vascular smooth muscle cells (VSMCs) and cardiac fibroblasts.

In some embodiments, the cell of the endoderm lineage may be a hepatic cell. The hepatic cell may be selected from the group consisting of hepatocytes, Kupffer cells, stellate cells and sinusoidal endothelial cells.

In one embodiment, the lineage specific cell is present in a homogenous population of cells. In another embodiment, the lineage specific cell is present in a substantially homogenous population of cells. For example, the substantially homogenous population of lineage specific cells may be at least about 60%, at least about 70%, at least about 80%, at least about 90% or about 100% homogenous.

In some embodiments, the homogenous population of lineage specific cells generated using the method of the present invention may be mixed. The mixture of homogenous populations may give rise to cellular networks, for example, a cortical network.

In some embodiments, the one or more reprogramming factors may be selected from the group consisting of a transcription factor, a chromatin remodeler, an epigenetic modifier and/or a non-coding RNA. The non-coding RNA may be microRNA. The transcription factor may be a neural transcription factor. In some embodiments, the neural transcription factor may be one or more transcription factors selected from the group consisting of Ngn1, Ngn2, Ngn3, Neuro D1, Neuro D2, Brn1m Brn2m Brn3A, Brn3B, Brn3C, Brn4, Dlx1, Dlx2, Asc11, phospho-dead mutant of the transcription factor Asc11 (SA/SV-Asc11), CTIP2, MYT1L, Olig1, Zic1 Nkx2.1, nkx2.2, Lhx2, Lhx3, Lhx6, Lhx8, SATB1, SATB2, Dlx5, Dlx6, Fezf2, Fev, Lmx1b, Lmx1a, Pitx3, Nurr1, FoxA2, Sox11, Atoh7, Olig2, Ptf1a, MEF2c, p55DD (dominant negative), Nkx6.1, Nkx6.2, Sox10, ST18, Myrf, Myt1, Zfp536, hes1, hes5, hes6, SOX2, SOX9, PAX6, NFIA, NFIB, NFIX, NICD, Islet1, Islet2, Irx3, Dbx2 and TAL1.

In a preferred embodiment, the one or more transcription factors are Asc11 (SA/SV-Ascl1) and Dxl12. In a further preferred embodiment, Ascii (SA/SV-Ascl1) and Dxl12 are linked by the T2A peptide. In another preferred embodiment, the transcription factor is NeuroD2.

The transcription factor may be a cardiac transcription factor. In some embodiments, the cardiac transcription factor is one or more transcription factors selected from the group consisting of Isl1, Mef2, Gata4, Tbx5, Nppa, Cx40, MESP1, MYOCD and ZFPM2, Baf60c, Hand2, Hopx, Hrt2, Pitx2c and nkx2.5.

In some embodiments, the transcription factor may be a hepatic transcription factor. The hepatic transcription factor may be one or more transcription factors selected from the group consisting of Hnf-1a, Hnf-1β, Hnf-3β, Hnf-3γ, Dbp, Lrh-1, Fxra, C/Ebpβ, Pxr, FOXA1, FOXA2, PROX1, HNF6, GATA6, PPARA, ZHX2, ONECUT2, ATF5, USF2, USF1, ZGPAT and NFIA.

In some embodiments, the microRNA is microRNA-9/9* and/or microRNA-124, miRNA-219, miRNA-338, miRNA-1, miRNA-133 and miRNA-187. In one embodiment, microRNA may be microRNA-9 and microRNA-124. In some embodiments, the one or more microRNAs may be linked to a reporter gene. In one embodiment, microRNA-9 and microRNA-124 are linked to a red fluorescent protein (RPF) gene.

The method as described herein may further comprise the step of contacting the population of non-lineage specific cells with an expression vector comprising a fluorescent indicator. The fluorescent indicator may be a calcium indicator, for example GCaMP6.

In another embodiment, there is provided a method of generating a directly convertible stem cell. In particular the method comprises the steps of: transfecting a stem cell with an expression vector comprising i) one or more cell lineage reprogramming factors operably linked to an inducible promoter and ii) a selection marker operably linked to a constitutive promoter; and screening the transfected stem cell for expression of the selection marker to generate said directly convertible stem cell, wherein said directly convertible stem cell is capable of direct conversion into an induced lineage specific cell.

In another embodiment, there is provided a directly convertible stem cell comprising i) one or more reprogramming factors operably linked to an inducible promoter and ii) a selection marker operably linked to a constitutive promoter, wherein said directly convertible stem cell is capable of direct conversion into an induced lineage specific cell.

In some embodiments, the directly convertible stem cell may be a cell line.

In another embodiment, there is provided a method of screening one or more factors and/or one or more genetic mutations that modulate a pre-selected activity of the induced lineage specific cell according to any one of the preceding claims, comprising the steps of: culturing said induced lineage specific cell in the presence of one or more factors and/or one or more genetic mutations; measuring the pre-selected activity of the lineage specific cell of step a); and comparing the measurement of b) relative to the measurement of the pre-selected activity in the lineage specific cell that has not been cultured in the presence of the said one or more factors or genetic mutations, wherein the difference in the measurement of the pre-selected activity of the lineage specific cell in c) indicates that the one or more factors or genetic mutations modulates the pre-selected activity of the lineage specific cell.

In some embodiments, the one or more factors may be selected from the group consisting of a drug, a growth factor, a small molecule, a biologic, a toxin, a stressor or a cell.

The one or more genetic mutations may be an engineered mutation or a naturally occurring mutation. In some embodiments, the engineered mutation may be selected from the group consisting of site-directed mutation, deletion, duplication, inversion, copy-number variation, imprinting and random mutation. The naturally occurring mutation may be a polymorphism selected from the group consisting of single nucleotide polymorphism (SNP), microsatellite variation, small-scale insertion/deletion and polymorphic repetitive element.

In some embodiments, the pre-selected activity of the lineage specific cell may be a genetic activity or susceptibility to a disorder. The susceptibility to a disorder may be determined by one or more intracellular or extracellular assays or combinations thereof, selected from the group consisting of Ca2+ imaging, cell survival, intrinsic firing properties, measurement of Na+channels, measurement of Ca2+ channels, measurement of K+ channels, synaptic activity, dendritic arborisation, axonal growth and targeting, neurotransmitter release and uptake, and intracellular Ca2+ activity.

In some embodiments, the genetic activity may be selected from the group consisting of gain-of-gene-function, loss-of-gene-function, gene knockdown, gene knockout and gene activation. The genetic activity may be achieved by small hairpin RNA (shRNA), small interfering RNA (siRNA) or CRISPR-associated (Cas) endonuclease.

In some embodiments, the disorder may be a neural disorder. The neural disorder may be selected from the group consisting of schizophrenia, autism, Alzheimer's disease, Parkinson's, Depression, ADHD, dementia, epilepsy, Huntington's, Angelman syndrome, motor neuron disease (MND) and Dravet syndrome. It will be generally understood that motor neuron disease encompass a group of diseases that includes but is not limited to amyotrophic lateral sclerosis (ALS), progressive bulbar palsy, pseudobulbar palsy, primary lateral sclerosis (PLS), progressive muscular atrophy, spinal muscular atrophy and post-polio syndrome (PPS).

In another embodiment, there is provided a kit for generating an induced lineage specific cell, comprising, a directly convertible stem cell as described herein; an inducer; and optionally instructions for use.

In another embodiment, there is provided a method of directly converting a stem cell into a lineage specific cell comprising: transfecting said stem cell with at least one expression vector comprising i) one or more cell lineage reprogramming factors operably linked to a constitutive promoter.

In some embodiments, the at least one expression vector may comprise a selection marker operably linked to a constitutive promoter. Alternatively, the selection marker may be operably linked to a constitutive promoter.

In another embodiment, there is provided a method of directly converting a stem cell into a lineage specific cell comprising: transfecting said stem cell with at least one expression vector comprising i) one or more cell lineage reprogramming factors operably linked to an inducible promoter.

In another embodiment, there is provided a stern cell directly convertible into a GABAergic neuron comprising: i) SA-ASCL1, DLX2, LHX6 and miR-9/9*-124 linked to a doxycycline inducible promoter; and ii) a selection marker operably linked to a constitutive promoter.

In another embodiment, there is provided a stem cell directly convertible into an excitatory neuron comprising: i) NeuroD2 linked to a doxycycline inducible promoter; and ii) a selection marker operably linked to a constitutive promoter.

In another embodiment, there is provided a method of screening an agent using a cell obtained by the method as disclosed herein comprising: i) contacting said cell with the agent; ii) measuring a pre-selected activity of the agent on the cell and comparing this to a cell that has not been contacted with the agent; and iii) detecting the activity of the agent on said cell.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

### BRIEF DESCRIPTION OF THE DRAWINGS

The invention will be better understood with reference to the detailed description when considered in conjunction with the non-limiting examples and the accompanying drawings, in which:

FIG. 1. Identification of genetic elements that efficiently convert hPSCs to induced GABAergic neurons (iGNs). (A) Schematic diagram illustrating expression of four genes (ASCL1, DLX2, NKX2.1, and LHX6) that are involved in genesis of cortical GABAergic neurons. D: dorsal. V: ventral. NCX: neocortex. LGE: lateral ganglionic eminence. MGE: medial ganglionic eminence. POA: Preoptic Area. (B) An overview for direct induction of hESCs to neurons. One day after plating as single cells, hESCs were lentivirally transduced with various combinations of reprograming factors. Neuronal conversion of hESCs was typically assessed at 10 dpt, and detailed characterization of converted neurons was performed at 42 dpt or later. dpt: days post transduction. mTeSR1: hPSC media. NM: neuronal media. (C) Efficiency of neuronal conversion of hESCs (line H1) at 10 dpt upon single transcription factor overexpression. The percentage of MAP2-positive cells over all cells (DAPI-positive) is shown (L, LHX6; N, NKX2.1; D, DLX2; A, ASCL1; A^{SA}, ASCL1 phosphomutant). Data are means +SEMs (n>3 independent experiments).

One-way ANOVA followed by Tukey's test shows significant differences, ***P<0.001. (D) Efficiencies of pan-neuronal conversion (black bars) and GABAergic neuronal conversion (white bars) of hESCs (line H1) upon indicated TF combinations at 10 dpt. Data are means±SEMs (n=3 independent experiments). The optimal combination, A^{SA}DL, is shown in red. (E) Efficiency of neuronal conversion of A^{SA}DL-converted hESCs transduced with or without miR-9/9*-124 (shown as miR for simplicity throughout the manuscript) at 10 dpt. Data are means±SEMs (n>3 independent experiments). Two-tailed, unpaired t-test shows significant difference, ***P<0.001. (F) Efficiencies of pan-neuronal conversion (black bars) and GABAergic neuronal conversion (white bars) of two hESC lines (H1 and H9) and three hiPSC lines (iPSC1-3) by A^{SA}DL+miR at 10 dpt. Data are means±SEMs (n=3 independent experiments). (G) Representative images of H1 derived induced GABA-positive neuronal cells (iGNs) immunolabeled with MAP2 and markers of neuronal subtypes (GABA, gamma-aminobutyric acid; TH, tyrosine hydroxylase; CHAT, cholineacetyltransferase; 5-HT, 5-hydroxytryptamine (also known as serotonin); VGLUT1/2, vesicular glutamate transporter 1 and 2). Data were collected at 42-50 dpt. Scale bar=20 μm. The percentage of neuronal subtype marker-positive cells that also express neuronal marker MAP2 is shown in (H). Data are means±SEMs (n=3 independent experiments). One-way ANOVA followed by Tukey's test shows significant differences, ***P<0.001.

FIG. 2. Expression of forebrain interneuronal markers in iGNs. (A) Quantitative RT-PCR using Fluidigm Biomark platform, performed on cytoplasm aspirated from single iGN cells via a patch pipette. iGNs derived from H1 hESCs by overexpressing A^{SA}DL+miR-9/9*-124 were collected at 48-52 dpt. Expression levels (shown as Ct values) are color-coded at the bottom. Genes analyzed are indicated on the right with their cellular functions. Numbers indicate individual iGN cells analyzed. (B) Immunostaining of iGNs with antibodies against NeuN (left, a mature neuronal marker), SMI-312 (middle, an axonal marker), and Ankyrin G (right, a marker for axon-initiating segment). Scale bar=20 μm. (C) iGNs expressed the telencephalic marker FOXG1 (left) but not DARPP-32 (right), a marker of striatal MSNs. Scale bar=20 μm. The insert inside the right panel showed positive signal of DARPP-32 antibody on cultured rat striatal neurons, which are known to contain DARPP-32 expressing MSNs. Insert scale bar=60 μm. (D) Left: representative image of iGNs stained with GAD1 antibody (GABA synthesis enzyme). Right: Confocal image of iGNs stained with antibodies against the vesicular GABA transporter VGAT, the inhibitory postsynaptic protein Gephryin, and the neuronal marker MAP2. Scale bar=20 μm. The inset was magnified on the right to show the juxtaposed bouton-like signals of VGAT and

Gephyrin illustrating morphological inhibitory synapses. (E) Immunostaining of iGNs with antibodies against interneuronal subtype markers, including somatostain (SST), calretinin (CR), calbindin (CB), and neuropeptide-Y (NPY), and parvalbumin (PV). Data were collected between 42-56 dpt, except for PV, which was collected at 70-90 dpt. Scale bar=20 μm. (F) Immunostaining of iGNs with antibodies against interneuronal subtype markers, including Reelin (RELN), neuronal nitric oxide synthetase (nNOS), and vasoactive intestinal peptide (VIP). Arrowheads point to neuronal cells that also expressed subtype makers as indicated. Scale bar=20 μm. (G) Quantification of percentage of interneuronal subtype markers shown in (E) and (F) as a bar graph. Data are means±SEMs (n=3 independent experiments).

FIG. 3. Electrophysiological properties of iGNs induced from hESCs. (A-F) Intrinsic electrophysiological properties of iGNs. (A) Representative traces showing the presence of voltage-dependent Na⁺ and K⁺ currents in iGNs. Blue box points to Natchannel dependent inward current. (B) Averaged (means±SEM) current-voltage relationship (I/V curves) for Na⁺ and K⁺ currents, recorded from iGNs at either 42 dpt (n=41) or 56 dpt (n=53). (C) Quantification of membrane resistance (Rm, left), membrane capacitance (Cm, middle), and resting membrane potential (RMP, right) recorded from iGNs at either 42 dpt (n=41) or 56 dpt (n=53). Statistical significance was assessed by two-tailed, unpaired t-test (*p<0.05, **p<0.01, ***p<0.001). (D) Representative traces of different patterns of spontaneous action potentials (APs), recorded from iGN at 56 dpt under cell-attached mode. Total number of cells analyzed: n=16. (E) Representative trace of multiple APs generation (the lower panel) recorded from iGN at 56 dpt triggered by current injection (the upper panel). (F) Characterization of APs generation properties in terms of spikes frequency with current-pulse amplitude, recorded from iGNs at either 42 dpt (n=41) or 56 dpt (n=53). (G-L) Demonstration of GABA release from iGNs. (G) Schematic diagram showing the patching on an induced excitatory neuron (iEN) which is synaptically connected with a ChETA-expressing iGN. (H) Merged differential interference contrast (DIC) and fluorescence (EYFP labels iGNs and turboRFP labels iENs) images of induced neurons for optogenetics. The image on the right depicts selecting patching on iEN, enlarged from the left image. Scale bar=5μm. (I) Upper panel: overlaid traces showing synaptic responses (upper panel) evoked by repeated presynaptic optogenetic stimuli (5ms every 30s, showed as blue light) in iGNs. Only 2 out of 10 stimuli failed to trigger IPSCs. Lower Panel: addition of bicuculline (20 μM) completely blocked the evoked response. (J) Exogenous GABA (1 mM, 100ms)-evoked response (black trace) recorded from iGN. This response was blocked by bicuculline treatment (red trace). (K) Representative traces of spontaneous IPSCs (sIPSCs) recorded in iGN, which are blocked by bicuculline. A blue box illustrated details of sIPSCs. (L) Quantification of sIPSC frequency (left) and amplitude (right) recorded from iGNs (n=61) at 42 and 56 dpt.

FIG. 4. Functional maturation and integration of transplanted human iGNs in the mouse cortex. (A) An overview to illustrate the dispersion of human iGNs (red) in the mouse cortex, mostly in layer 5/6. Scale bar=100 μm. The white box is magnified three times and is shown in (B). (B) An illustration of the neurite arborization of the human iGNs (red). Human iGNs also expressed the mature neuronal marker NeuN. Scale bar=50 μm. (C) Transplanted human iGNs expressed the pan-neuronal marker MAP2, GABAergic markers GAD67 and GABA, and interneuron subtype marker SST. Scale bar=20 μm. Quantifications of the percentage of human cells positive for each marker is shown in (D). Data were obtained from n=3 animals, with more than 20 cells counted for each marker. (E) Representative trace of multiple APs generation (the lower panel) recorded from transplanted human iGN triggered by current injection (the lower panel). (F) Characterization of APs generation properties of human iGNs in terms of spikes frequency with current-pulse amplitude. (G) Representative traces of spontaneous EPSCs (sEPSCs) recorded in iGN, which are blocked by CNQX (50 μM). A blue box illustrated details of sEPSCs. (H) Quantification of sEPSC frequency (left) and amplitude (right) recorded from iGNs (n=9). Data are shown as the mean±SEMs.

FIG. 5. Applications of iGNs in studies of neural network activity and in functional interrogation of gene specifically affecting inhibitory synapses. (A) Schematic diagram illustrating co-culture of human induced excitatory (iEN, 80%) and inhibitory neurons (iGN, 20%) to mimic the ratio found in mammalian cortex. iGNs were generated by overexpression of A^{SA}DL+miR-9/9*-124 and iENs were generated by overexpression of NeuroD2 (see also Supplementary Materials and Methods). (B) Both sIPSCs and sEPSCs were recorded from iGNs cultured on coverslips containing mixed iENs/iGNs, which are blocked by bicuculline and CNQX, respectively. Data were collected at 56 dpt. (C) Zoomed-in traces for boxes 1-3 in (B). (D-E) Representative raster plots of calcium spikes in iENs (D) or mixed iENs/iGNs network (E) with addition of bicuculline (indicated in red) after 2 minutes of baseline recording. (F) Statistical analyses of bursts number (top) and synchronization index (bottom) of iENs (left) or mixed iENs/iGNs cultures (right) (n=5 and 4 for iENs and mixed iENs/iGNs, respectively). (G) Representative images of VGAT immunofluorescence staining of iGNs with control or MDGA1 overexpression. Scale bar=5 μm. (H) Quantification of VGAT positive bouton density, demonstrated by number of boutons over area of randomly chosen regions (left) with similar arborization of dendrites (right) (n=8 for both control and MDGA1 overexpression). (I) Representative traces of sIPSCs recorded in iGNs with control (left) or MDGA1 (right) overexpression.

(J-K) Cumulative plots and histograms of sIPSCs frequency (J) and amplitude (K) recorded in iGNs with control or MDGA1 overexpression (n=13 and 12 for control and MDGA1 overexpression, respectively). All histogram data are shown as the mean±SEMs. Statistical significance was assessed by two-tailed, unpaired t-test except for (F), two-tailed, paired t-test, (*p<0.05).

FIG. 6. Characterization of hPSC lines by immunostaining with specific antibodies against pluripotency markers and neural progenitor markers. Two hESC lines (H1 and H9) and three human iPSC lines (hiPSC1-3) expressed pluripotency markers including OCT4, NANOG, and SOX2, but did not express neural progenitor markers NESTIN and MUSASHI. Two insets: images of human neural progenitor cells that were immunostained with specific antibodies against NESTIN (the left bottom inset, red) and MUSASHI (the right bottom inset, green) as positive controls, Scale bar=20 μm.

FIG. 7. Identification of genetic elements for efficient differentiation of GABAergic neurons (iGNs) from hPSCs and molecular characterization of resulting iGNs. (A) Quantification of neuronal conversion efficiency by combinations of multiple transcription factors, represented by the percentage of MAP2-positive cells over DAPI positive signals. Note that combinations of D, N, and L without A could not produce significant number of neurons. Data collected at 10 dpt. Data are means±SEMs (n=4 independent experiments). One-way ANOVA followed by Tukey's test shows significant differences, ***P<0.001. (B) Representative images of hESCs transduced with A^{SA}DL factors either with or without miR-9/9*-124 (shown as miR for simplicity throughout the manuscript) at 10 dpt. Scale bar=20 μm. Efficiency of neuronal conversion is shown in

(H). Data are means±SEMs (n=3 independent experiments). Two-tailed, unpaired t-test shows significant difference, ***P<0.001. (C) Quantification of total dendritic length (left) and primary branch number (right) based on analysis of MAP2 staining in A^{SA}DL or A^{SA}DL+miR transduced H1 hESCs at 10 dpt. Between 60-80 cells were analyzed in each case. Two-tailed unpaired t-test shows significant difference, *P<0.05, ***P<0.00. (D) Quantitative RT-PCR analysis showed that iGNs (transduced by A^{SA}DL+miR-9/9*-124 express genes required for GABA transport and synthesis (VGAT, GAD1 and GAD2) at 14 dpt, and the expression of all three genes robustly further increased at 35 dpt. Results are shown as normalized to HN (fetal human neurons, purchased from Sciencell), which contained about 10-15% of GABAergic neurons and served as positive controls. (E) Immunostaining images showed that some iGNs expressed interneuron subtype markers SST and CR respectively, while some cells expressed both markers. Scale bar=20 μm.

FIG. 8. Electrophysiological characterization of iGNs. (A-D) Firing properties of iGNs converted from additional hPSCs cell lines. (A) Representative traces of multiple APs generation (the upper panel) recorded from an iGN (derived from H9 hESCs) at 50 dpt triggered by current injection (the lower panel). (B) Quantification of APs generation properties in terms of spikes frequency with current-pulse amplitude, recorded from iGNs (derived from H9 hESCs) at 50 dpt (n=12). (C) Representative traces of multiple APs generation (the upper panel) recorded from an iGN (derived from hiPSC #1) at 50 dpt triggered by current injection (the lower panel). (D) Quantification of APs generation properties in terms of spikes frequency with current-pulse amplitude, recorded from iGNs (derived from hiPSC #1) at 50 dpt (n=13). (E-H) Characterization of four different AP firing patterns of iGNs recorded at 42-56 dpt. (E) Representative traces of four different AP firing patterns: accommodation, non-accommodation, anti-accommodation and single-spike, recorded in iGNs at 42-56 dpt. (F) A pie chart illustrating the proportion of each firing pattern observed (total n=91) at 42-56 dpt iGNs. (G) Quantification of action potential (AP) threshold (left), AP half width (middle) and after-hyperpolarization (AHP, right) of iGNs (42-56 dpt) exhibiting accommodation (type I) or non-accommodation (type II) firing patterns. Statistical significance was assessed by two-tailed unpaired t-test, **p<0.01. (H) Characterization of spikes frequency with current-pulse amplitude of iGNs exhibiting accommodation (type I) or non-accommodation (type II) firing patterns. All data are shown as the meant SEMs.

FIG. 9. Molecular and electrophysiological characterization of human induced excitatory neurons (iENs). (A) Quantitative RT-PCR analysis showed that iENs (differentiated by forced ectopic expression NeuroD2 in hESCs) express genes responsible for glutamate transport VGLUT1 and VGLUT2 at the high level, but did not express those genes that were involved with GABA transport and synthesis (VGAT, GAD1 and GAD2) at 35 dpt. Results shown were normalized to level of gene expression in HN (fetal human neurons, purchased from Science11, served as positive controls). (B-G) Electrophysiological properties of iENs at dpt 21. (B) Averaged current-voltage relationship (I/V curves) for Na⁺ and K⁺ currents (n=34). (C) Representative trace of multiple APs generation (the upper panel) recorded from iEN triggered by current injection paradigm (the lower panel). (D) Characterization of AP spiking frequency with current injection steps (n=34). (E) Representative traces of spontaneous EPSCs (sEPSCs) recorded from iENs at 21 dpt. A zoomed box (blue) was used to illustrate detailed traces of sEPSCs. (F) Application of CNQX (20 μM) completely abolished sEPSCs in iENs at 21 dpt, indicating that most of synaptic transmission between iENs were excitatory glutamatergic synaptic transmission. (G) Quantification of sEPSCs frequency (left) and amplitude (right) recorded from iENs.

FIG. 10. Identification of optical stimulus evoked response of ChETA-expressing iGNs induced from hESCs. (A) Schematic diagram showing the whole-cell patch clamp recording on induced iGNs expressing ChETA. (B) Inward currents triggered by optical stimuli under voltage clamp (the upper panel) recorded in an iGN expressing ChETA at 56 dpt. Application of a sodium channel blocker TTX (1 μM) could not block these light-evoked currents (the lower panel). (C) Inward currents induced by light stimulation with varying intensity (0.25 mW to 1.00 mW). (D) APs triggered by light stimulation (1 Hz) under current clamp mode. (E) A schematic diagram showing the whole-cell patchclamp recording on induced iGNs without ChETA expression. (F) No inward current was recorded upon light stimulation in iGNs without ChETA expression.

FIG. 11. Electron microscopy of iGNs transplanted into mouse cerebral cortex. (A-C) Three consecutive serial images (1-3) demonstrating ultrastructure of RFP/DAB-stained iGN dendrites (labeled as D, yellow) that make synapses (arrowheads) with a presynaptic bouton (yellow asterisks). Scale bar=1 μm.

FIG. 12. MDGA1 overexpression in iENs did not alter excitatory synapses formation. (A) Representative traces of sEPSCs recorded in iENs with control (left) or MDGA1 overexpression (right). (B-C) Cumulative plots and histograms of sIPSCs frequency (B) and amplitude (C) from iENs with control (grey) or MDGA1 overexpression (black) (Control: n=11; MDGA1 overexpression: n=10). All data were collected from 21 dpt iENs derived from H1 hESCs. Statistical significance was assessed by two-tailed unpaired t-test.

FIG. 13. Dox-inducible iGN hESC line. (A) Differential interference contrast (DIC) images illustrating the morphological changes of inducible-iGN hESC line (derived from H1) at the indicated days upon doxycycline (Dox, 1 μg/ml) treatment. Scale bar=40 μm. (B) Immunostaining of Dox-inducible iGNs with specific antibodies against neuronal markers MAP2 (red) and NeuN (green) at the indicated days upon Dox treatment. Scale bar=20 μm.

### EXPERIMENTAL SECTION

Non-limiting examples of the invention, including the best mode, and a comparative example will be further described in greater detail by reference to specific Examples, which should not be construed as in any way limiting the scope of the invention.

Materials and Methods

Generation of Plasmid Constructs

A bi-cistronic lentiviral backbone as described (addgene #31780) was used to clone cDNAs encoding hASCL1 (NM_004316.3), hASCL1-phosphmutant, hNKX2.1 (NM_003317.3), hDLX2 (NM_004405.3), hLHX6 (NM_014368.4), and hNeuroD2 (addgene #31780) under the EF1 a promoter, respectively. Doxycycline (Dox)-inducible lentiviral miR-9/9*-124 construct was as described (addgene #31874). Lentiviral expression vector of rtTA was modified from addgene #20342 (FUW-rtTA-pGK-hygromycin).

Upon identification of optimal genetic elements (A^{SA}DL+miR-9/9*-124), four reprogramming factors were packed into two dox-inducible lentiviral constructs (TetO-A^{SA}-T2A-DLX2-pGK-blastsicidin, modified from addgene #27151, and pTight-hLHX6-miR-9/9*-124-IRES-puro, modified from addgene #31874).

For optogenetic experiments, cDNA encoding ChETA-EYFP was obtained from Addgene #26967 and subcloned into a lentiviral construct with a human synapsin promoter (Synapsin-ChETA-EYFP). For MDGA1 overexpression, cDNA encoding human MDGA1 was subcloned to lentiviral backbone of addgene #20342 (FUW-MDGA1).

To generate lentiviral particles, lentiviral expression vectors together with psPAX2, and pMD2.G were co-transfected into Lenti-X 293T cells (Clontech) using Fugene HD (Roche). Supernatants were collected from culture media and lentiviral particles were concentrated using a PEG-it kit (# LV810A-1, System Biosciences), following manufacturer's protocol.

hPSCs Cell Culture

Human ESC lines H1 and H9 were originally obtained from WiCell Research Institute (Madison, Wis.), and have been maintained in the laboratory. hiPSC line #1 was obtained from Kerafast (AG1-0). hiPSC line #2 and #3 were GM23338 and GM23279, respectively, both purchased from Coriell Institute. All lines (hESCs, hiPSCs, and iGN inducible lines) were cultured in mTeSR1 media under feeder-free conditions in matrigel-coated, cell culture plates and are routinely passaged (1:6 to 1:10) using Dispase or ReLeSR (all from Stemcell Technologies). All lines used displayed normal karyotypes.

Generation of induced neuronal cells from hPSCs

hESCs and hiPSCs were dissociated with TrypLE (Life Technologies) to single cells and plated onto matrigel coated cell culture plates in mTeSR1 media supplemented with thiazovivin (1 μM, TOCRIS). On the following day, cells were transduced with lentiviruses expressing various transcription factors and microRNAs as indicated in the study, and this was designated as day 0. Next day (1 dpt), culture media was completed changed Neuronal Media (Sciencell), which was used until the end of the experiments. For experiments using Dox-induced iGN lines, doxycycline was added into the media at 1 μg/ml, and maintained for 3 weeks. Cells were selected with appropriate antibiotics from 3-7 dpt to enrich transduced cells. For molecular and functional characterization of induced neurons (dpt 14 or later), cells were dissociated at 7-10 dpt and replated onto poly-L-lysine/laminin-coated glass coverslips or onto cell culture plates. Primary rat glial cells (derived from P1 neonatal rat cortices, cultured more than 2 passages in vitro) and neurotrophic factors (BDNF, GDNF, NT3, and IGF1, each at 10 ng/ml, and all from Peprotech) were added to human induced neuronal cells at 14-20 dpt to enhance survival and synaptic maturity of the induced neurons.

For the generation of inducible iEN/iGN hESC line, H1 hESC stably expressing rtTA was first established (lentiviral transduction with hUbc-rtTA-pGK-hygro and subsequently selected with hygromycin). This line was further transduced with TetO-hNeuroD2-pGK-puro and selected with puromycin to generate the inducible iEN line. Similarly, we generated dox-inducible iGN line by transducing the rtTA-expressing H1 line mentioned above with TetO-ASA-T2A-DLX2-pGK-blasticidin and TetO-miR-9/9*-124-pGK-puromycin, and stably selected with blasticidin and puromycin.

Fluorescent Immunocytochemistry (Cultured Cells)

Immunostaining experiments were performed. Cells were fixed in 4% PFA for 20 minutes, and permeabilized with 0.2% triton X-100 for 15 minutes before blocking for 60 minutes. Blocking buffer was made up of 5% BSA and 2% FBS in PBS. Primary antibodies were diluted in blocking buffer and incubated with cells overnight at 4 degrees. The secondary antibodies were donkey or goat anti-rabbit, mouse, or chicken IgG conjugated with Alexa-488, -594, or -647 (Invitrogen). The following primary antibodies were used: chicken anti MAP2 (Abeam AB5392), mouse anti MAP2 (Abcam AB112670), mouse anti beta III tubulin (Covance, MMS-435P), mouse anti NeuN (Millipore MAB377), mouse anti Ankyrin G (NeuroMab 75-146), mouse anti SMI-312 (Covance SMI-312R), rabbit anti FOXG1 (Abeam AB18259), mouse anti Reelin (Millipore MAB5364), goat anti ChaT (Millipore AB144P), guinea pig anti VGLUTI (Millipore AB5905), guinea pig anti VGLUT2 (Millipore AB5907), rabbit anti DARPP-32 (Santa Cruz sc-11364), mouse anti Gephyrin (SYSY 147021), rabbit anti Synapsin (Millipore MAB355), rabbit anti nNOS (Immunostar, 24287), mouse anti GAD1 (Millipore MAB5406), rabbit anti VGAT (SYSY 131003), rabbit anti NPY (Abeam 10980), rabbit anti VIP (Immunostar 20077), mouse anti PV (SWANT, PV235), goat anti Calretinin (Millipore MAB1550), mouse anti Calbindin 1(SWANT 300), rabbit anti SST (Peninsula/Bachem T4103), rabbit anti GABA (Sigma A2052), mouse anti TH (Immunostar 22941), rabbit anti 5-HT (Immunostar 20080). Images were acquired using Observer Z.1 or LSM 710 (Zeiss). VGAT and gephyrin boutons were analyzed with MetaMorph (Universal Imaging) and Image J (National Institutes of Health). Areas with similar density of neurons were randomly chosen for analyses of VGAT density. VGAT fluorescence signals that were less than 0.22 μm2 (in area) were excluded from analyses. Same intensity threshold was used for both control and MDGA1 overexpression neurons. Total density length was quantified using MAP2 signal in each chosen area to confiini the VGAT density calculation is reliable by respecting to image area.

Gene Expression Analyses

For quantitative RT-PCR analyses of pooled cultured cells, RNA was extracted using DirectZol (Zymo), treated with DNAse, and converted to cDNA using High Capacity cDNA Reverse Transcription kit (Life Technologies). Real-time PCR assay was performed using the Applied Biosystems 7900HT Fast real-time PCR system. Multiplex Single cell qPCR was performed. Cytoplasm of single induced neuronal cells (7 weeks after transduction) growing on coverslips was aspirated into patch pipette and ejected into 2× cells-direct buffer (Life Technologies), flash-frozen, and kept at −80° C. until processing. Thawed cytoplasm was subjected to reverse transcription (Superscript III, Life Technologies) and 18 cycles of PCR pre-amplification with pooled primers specific to the target genes (STA). Unused primers were then digested away using Exonuclease I (New England BioLabs, PN M0293). The cleaned-up cDNA of individual cells was processed for real-time PCR analysis on Biomark 96:96 Dynamic Array (Fluidigm) on a Biomark HD System (Fluidigm) using the indicated primer pairs (FIG. 2, See Table 1 for complete primers information) and SsoFast EvaGreen Supennix with Low ROX (Bio-Rad), following manufacture's protocol. Data was collected and analyzed using the Fluidigm Real-Time PCR Analysis software. Results were represented as a heatmap using color-coded Ct values as indicated in FIG. 2A. Primers used were tested prior to ensure specific melting curves and additionally validated by using human cDNA library prepared from human neurons (ScienCell). The aspirated cells were confirmed based on GAPDH expression.

TABLE 1
Primers used in gene expression analyses.

| Gene | | |
|---|---|---|
| Symbol | Forward (5′-3′) | Reverse (5′-3′) |
| ANK2 | TCACAACTGAGTCATCCATCAC | ATTACCTGCGATACAGCTTGG (SEQ |
| | (SEQ ID NO: 1) | ID NO: 2) |
| COUPTF1 | CTCAAGAAGTGCCTCAAAGTG | AGATGTAGCCGGACAGGTAG (SEQ |
| | (SEQ ID NO: 3) | ID NO: 4) |
| DARPP-32 | TCTCAAGTCGAAGAGACCCAAC | TGCAGGTGAGACTCAGCAA (SEQ ID |
| | (SEQ ID NO: 5) | NO: 6) |
| DAT | AGAGACGAAGACCCCAGGAAGT | GAGGTTGAAGAGTAGAAGTTGCCC |
| | (SEQ ID NO: 7) | T (SEQ ID NO: 8) |
| DLX1 | GGCTGTTTGCCAATTCAGGG (SEQ | CTCCCCGTGCGCTTAAAGTA (SEQ |
| | ID NO: 9) | ID NO: 10) |
| DLX5 | ACAGAGACTTCACGACTCCCAG | TGTGGGGCTGCTCTGGTCTA (SEQ |
| | (SEQ ID NO:11) | ID NO: 12) |
| DLX6 | TGGTGAAAGAGAAGCATTTTGGAC | AGAGAAGGGCTGTTATGTGAGGAA |
| | T (SEQ ID NO: 13) | (SEQ ID NO: 14) |
| EN1 | TCTCGCTGTCTCTCCCTCTC (SEQ | CGTGGCTTACTCCCCATTTA (SEQ |
| | ID NO: 15) | ID NO: 16) |
| ERBB4 | TCGTGTCGCCGCTTCAGTA (SEQ ID | GAGCCATTCTCAAACTCCCGAAA |
| | NO: 17) | (SEQ ID NO: 18) |
| FOXG1 | AGCCGCCAGATTTCCATGTGT (SEQ | CTCAAGGTCTGCGTCCACCA (SEQ |
| | ID NO: 19) | ID NO: 20) |
| GAD1 | CAAACATTTATCAACATGCGCTTC | CTATGACACTGGAGACAAGGC (SEQ |
| | (SEQ ID NO: 21) | ID NO: 22) |
| GAD2 | ATCCTGGTTGACTGCAGAGAC | CCAGTGGAGAGCTGGTTGAA (SEQ |
| | (SEQ ID NO: 23) | ID NO: 24) |
| GAPDH | CTCTGCTCCTCCTGTTCGAC (SEQ | GCGCCCAATACGACCAAATC (SEQ |
| | ID NO: 25) | ID NO: 26) |
| GFAP | CGGATCACCATTCCCGTGCA (SEQ | TTGAGGTGGCCTTCTGACACAG |
| | ID NO: 27) | (SEQ ID NO: 28) |
| GPHN | CACCAGAATTCGCACTTCAAG | AGACAGTAATGCCAGGACAAG |
| | (SEQ ID NO: 29) | (SEQ ID NO: 30) |
| GRIA1 | CTTCCCGGACCAAAGTGATAG | TGATGGAAAATACGGAGCCC (SEQ |
| | (SEQ ID NO: 31) | ID NO: 32) |
| GRIA2 | TGGAGTGAAGTGGACAAAATGGTT | GTCTTATTCTCAAGCCCAGAGGTGT |
| | G (SEQ ID NO: 33) | (SEQ ID NO: 34) |
| GRIN1 | AGGAACCCCTCGGACAAGTT (SEQ | CCGCACTCTCGTAGTTGTG (SEQ ID |
| | ID NO: 35) | NO: 36) |
| GRIN2A | GGGCTGGGACATGCAGAAT (SEQ | CGTCTTTGGAACAGTAGAGCAA |
| | ID NO: 37) | (SEQ ID NO: 38) |
| GRIN2B | GTAGCCATGAATGAGACCGAC | GGATCGGGGTGAGAGTCTGT (SEQ |
| | (SEQ ID NO: 39) | ID NO: 40) |
| GRP | ACCGTGCTGACCAAGATGTA (SEQ | GCTCCCTCTCTCAGAAACAGAA |
| | ID NO: 41) | (SEQ ID NO: 42) |
| HPRT1 | GCTTTCCTTGGTCAGGCAGTA (SEQ | ACTTCGTGGGGTCCTITTCAC (SEQ |
| | ID NO: 43) | ID NO: 44) |
| MAP2 | CAACGGAGAGCTGACCTCA (SEQ | CT ACAGCCTCAGCAGTGACTA |
| | ID NO: 45) | (SEQ ID NO: 46) |
| MAPT | GAAGATTGGGTCCCTGGACAATA | AGGTCAGCTTGTGGGTTTCA (SEQ |
| | (SEQ ID NO: 47) | ID NO: 48) |
| NANOG | GGACACTGGCTGAATCCTTCCT | CTCCAACCATACTCCACCCTCC |
| | (SEQ ID NO: 49) | (SEQ ID NO: 50) |
| NCAM | GGCTCCTTGGACTCATCTTTC (SEQ | GACATCACCTGCTACTTCCT (SEQ |
| | ID NO: 51) | ID NO: 52) |
| NESTIN | GGCTGAGGGACATCTTGAG (SEQ | TGCGGGCTACTGAAAAGTTC (SEQ |
| | ID NO: 53) | ID NO: 54) |
| OCT4 | AGGTGTCGGAATGCGTGGTG (SEQ | AATCCGGGAGCGGGCTTCTA (SEQ |
| | ID NO: 55) | ID NO: 56) |
| OLIG2 | CCCCACCGACTCATCTTTCCT (SEQ | CCAACAGAACCCCCAAATAACCC |
| | ID NO: 57) | (SEQ ID NO: 58) |
| PCP2 | CGGGCCAGACCACCAA (SEQ ID | TGTCACACGTTGGTCATCCA (SEQ |
| | NO: 59) | ID NO: 60) |
| PROX1 | CCTCCCATTACTCAGACCCGTG | AGGCTCCCGCTTAGAAACTGT (SEQ |
| | (SEQ ID NO: 61) | ID NO: 62) |
| PSD95 | AGCTGGAGCAGGAGTTCAC (SEQ | ACACGCTTCACCTTGTGGTA (SEQ |
| | ID NO: 63) | ID NO: 64) |
| SATB1 | AGAGCTGTCAGTGGAAGGAAACA | GTGGCACTGTTGAACGAAACAAAT |
| | (SEQ ID NO: 65) | (SEQ ID NO: 66) |
| SCN1A | AGAGGGCGAGCAAAGGATGTG | CGTCGGGGCACAAACAAGGA (SEQ |
| | (SEQ ID NO: 67) | ID NO: 68) |
| SERT | TGCTGGCTTTTGCTAGCTAC (SEQ | GAAGCTCGTCATGCAGTTCA (SEQ |
| | ID NO: 69) | ID NO: 70) |
| SOX2 | ATCCCATCCACACTCACGCAA | ACCCTCCCCAGGTTTTCTCTG (SEQ |
| | (SEQ ID NO: 71) | ID NO: 72) |
| SOX6 | AGTTCCTCTGGCTGACTCTATCTGT | AAACCTCACTGCTTCCCACCC (SEQ |
| | (SEQ ID NO: 73) | ID NO: 74) |
| SP8 | TGCTTGCTCCCGAATCAGACG (SEQ | TCTTCCTGGCACCCCCAACA (SEQ |
| | ID NO: 75) | ID NO: 76) |
| SYN1 | ATGTCCTGGAAGTCATGCTG (SEQ | CCCCAATCACAAAGAAATGCTC |
| | ID NO: 77) | (SEQ ID NO: 78) |
| TPH1 | CTTGGGAGAATTGGGCAAAAC | GAGACACAGTTCAGATCCCTTC |
| | (SEQ ID NO: 79) | (SEQ ID NO: 80) |
| TPH2 | ATGGCTCAGATCCCCTCTACA (SEQ | GGATCCGCAAGTAGTGGAACA |
| | ID NO: 81) | (SEQ ID NO: 82) |
| VGAT | AGATGATGAGAAACAACCCCAG | CACGACAAGCCCAAAATCAC (SEQ |
| | (SEQ ID NO: 83) | ID NO: 84) |
| VGLUT1 | TCAAGTCCCCGATTCCGTGC (SEQ | TGCGATTTTGGF1GTTTCCCCA (SEQ |
| | ID NO: 85) | ID NO: 86) |
| VGLUT2 | TGGGGCTACATCATCACTCA (SEQ | GAAGTATGGCAGCTCCGAAA (SEQ |
| | ID NO: 87) | ID NO: 88) |
| ZEB2 | GGGCATATCTGTGTCCCAATCCA | CACCCTCCTCTACTTCGTGCA (SEQ |
| | (SEQ ID NO: 89) | ID NO: 90) |

Electrophysiology

Whole cell patch clamp recordings were performed on iGNs and iENs with voltage or current clamp mode. Recording pipettes with resistances of 4-6 MΩ were filled with an internal solution containing (in mM): 120 K-gluconate, 9 KCl, 10 KOH, 3.48 MgCl2, 4 NaCl, 10 HEPES, 4 Na2ATP, 0.4 Na3GTP, 17.5 Sucrose, 0.5 EGTA with an osmolarity of 290 mOsm and pH 7.3. Neurons were bathed in the extracellular solution containing (in mM): 124 NaCl, 3 KCl, 1.3 NaH2PO4, 10 dextrose, 2 MgCl2, 2 CaCl2, 10 HEPES at pH 7.4. Bicuculline (20 μM, Tocris) and CNQX (50 μM, Tocris) were used to block inhibitory or excitatory synaptic responses, respectively. Neurons were held at −70 mV except otherwise indicated or at required currents using an Axon MultiClamp 700B amplifier (Axon Instruments). Signal was sampled at 40 kHz and filtered at 2 kHz (Digidata 1440A, Molecular Devices). Recordings with serial resistance higher than 20 MO or leaking current more than 200 pA were not analyzed. Data were analyzed offline using the ClampFit (Molecular Devices) or MiniAnalysis (Synaptosoft). Voltage clamp was used for recording I/V curve of Na/K current using a protocol of increasing holding potential 10 mV for each step from −80 mV. Membrane potential of patched neurons was held at −70 mV for spontaneous synaptic current. The reversal potential for ions was −49 mV in our recording system, calculated using the simplified Nernst equation: E(Cl⁻)=−59*log(extracellular[Cl⁻]/intracellular[Cl^{-]),(T=}25° C.). Spontaneous action potentials were recorded at I=0 mode. Action potentials also induced by current injection with steps at 10 pA lasting 800 ms after manually adjusting the membrane potential around −70 mV by current injection under current clamp mode. For cell-attached recordings, pipettes with resistances of 2-4 MΩ were filled with extracellular solution described above. Attachment between pipettes and neuron membrane was formed with 50-200 MΩ seal resistance. Voltage clamp mode was used to record current response by spontaneous spikes firing with holding pipettes at 0 mV.

Optogenetic or Chemical GABA Evoked IPSC Recording

iGNs that priorly transduced with Synapsin-ChETA-EYFP were co-cultured with iENs expressing turboRFP (FUW-tRFP). For optogenetical evoked IPSC recording, two nearly neurons with EYFP and tRFP, respectively, were visually identified with fluorescent microscope (Olympus) with DIC. Patch clamp recordings were performed on tRFP-positive neurons for evoked IPSC or on EYFP-positive neurons for identification of optical stimulation mediated by ChETA. Optical stimuli (5 ms duration, 30s interval) were provided with blue (470 nm) LED (Thorlabs, M470F1) controlled by digital input from Digidata 1440A (Molecular Devices).

For chemical GABA evoked IPSC recording, tip of glass pipettes filled with freshly made GABA (1 mM, Sigma) was put approximately 100 μm away from soma along dendrites of recorded neurons. Air puff to trigger GABA release was provided with PICOSPRITZER III (Parker) controlled by Digidata 1440A.

Transplantation

Immunodeficient NOD scid gamma (NSG) mice used for transplantation studies and breeding were a kind gift from Dr. David Virshup at Duke-NUS Graduate Medical School. Mice were housed in a specific pathogen free environment, maintained under 22° C., 55% humidity, with food and water provided ad libitum, on a 12-hr light/dark cycle (lights on at 0700 h). All procedures followed national guidelines for the care and use of laboratory animals for scientific purposes with approved protocols from the Institutional Animal Care and Use Committee of Duke-NUS Graduate Medical School.

P1 NSG pups were anesthetized on ice for 1-2 minutes before being secured with tape onto a prechilled ice block. Human iGNs were priorly labeled with RFP as described, and trypsinized to single cells at 8 dpt. Concentrated cell suspensions (˜2.5-5×10⁴ cells/μl) were front loaded into a microliter syringe (26s gauge, Hamilton Company) and injected (200 nl, 250 nl/min) bilaterally to a depth of 0.2 mm near the centre of the anterior/posterior axis and 1 mm away from the midline.

Slice Recording

After isofluorane anesthesia, brains were quickly cut out and 300-μm coronal slices were sectioned using vibrating microtome (VF-200 Microtome, Precisionary Instruments). Slices were incubated at 30° C. in artificial cerebrospinal fluid (ACSF) containing (in mM): 125 NaCl, 2.5 KCl, 1.25 NaH₂PO₄, 2 CaCl₂, 1 MgCl₂, 26 NaHCO₃, and 10 glucose, saturated with 95% 0₂ and 5% CO₂ at 30-32° C. for recovery for at least 1 hour. During recording, slices were kept in a recording chamber with continuous perfusion of ACSF. CNQX (50 μM, Tocris) was used to block sEPSC. Patch recordings were done by using IR-DIC visualization techniques with an Olympus BX51WI upright microscope with a ×60 water-immersion lens. Patch clamp recording method was same with that of recording in cultured cell.

Fluorescent Immunocytochemistry (Brain Slices)

2 months post transplantation, mice were transcardially perfused with ice cold PBS followed by 4% PFA (Sigma) in 0.1M PBS. Brains were dissected, post-fixed in 4% PFA overnight and cryoprotected with 30% sucrose in 0.1M PB until they sunk. Sections 30 μm thick were cut on a sliding microtome (Leica), washed with PBS, permeabilized with 0.2% Triton-X in PBS for 10 minutes, blocked in PBS with 2% BSA (Sigma), 5% donkey serum (Invitrogen) and 0.2% Triton X-100 at room temperature for 1 hour, and subsequently incubated with primary antibodies overnight. Following incubation, sections were washed three times with 0.2% Triton-X, and incubated for 2 hours at room temperature with goat anti-rabbit, mouse or chicken IgG conjugated with Alexa-488 or 647 (Invitrogen). Sections were washed once with 0.2% Triton-X, incubated with DAPI (Life-Technologies) for 10 minutes followed by two additional washes. Sections were mounted on glass slides with Fluor Save (Millipore).

Electron Microscopy

Mice were anesthetized and intracardially perfused with 2% paraformaldehyde and 2.5% glutaraldehyde in 0.15M cacodylate buffer. The brain was extracted from the skull and then postfixed overnight in the same fixative. Cortical tissues transplanted with RFP-expressing iGNs were sliced using a vibratome (˜100 μm). After targeting the location of fluorescent signals under a epifluorescent microscope, cortical slices were immunostained with a rabbit polyclonal anti-RFP antibody (MBL, 1:500), a biotinylated goat anti-rabbit secondary antibody, and the ABC-peroxidase kit (Vector Labs) and developed with DAB and hydrogen peroxidase. The slices were further prepared for serial block face-SEM (SBF-SEM) observation. Briefly, small pieces of immunostained slices were postfixed in 2% osmium tetroxide containing 1.5% potassium ferrocyanide and 2 mM calcium chloride in 0.15M cacodylate buffer, and then incubated in thiocarbohydrazide solution. Following the second exposure to 2% osmium tetroxide, tissue samples were en bloc stained in 1% uranyl acetate (Ted Pella), incubated in the lead aspartate solution, and then dehydrated in an ascending series of ethanol solutions. Samples were transferred to acetone and flat-embedded in Epon-812 (EMS). Epon-embedded specimens containing DAB-labeled neurons were glued on an aluminum stub (Gatan), painted with colloidal silver paste (Ted Pella), and then sputter-coated with gold/palladium to reduce charge artifacts. 11 stacks of serial images (tens to hundreds of 30-nm-thick sections/stack) were obtained using a scanning electron microscope (Merlin VP, Carl Zeiss NTS GmbH, Oberkochen, Germany) combined with the Gatan 3View2 diamond knife cutting system at the accelerating voltage of 1.5 kV. To cover the large area, low magnification images of the sample block were acquired with the both back-scattered and secondary electron detectors of the column.

Calcium Imaging

Neurons were incubated in extracellular solution containing (in mM): 124 NaCl, 5 KCl, 1.3 NaH2PO4, 10 dextrose, 2 MgCl2, 4 CaCl2, 10 HEPES at pH 7.4 with Fluo-4 AM (2 μM, ThermoFisher Scientific, F-14201) in incubator (37oC, 5% CO2) for 30 mins before acquiring images. Imaging solution was identical to extracellular solution. Live images were acquired with an LSM 710 (Zeiss) confocal microscopy using 20x objective at 1 Hz at 37° C. Calcium spikes sorting and analysis was done referred to previous works ^{6,7}. Calcium spikes were identified with more than 20% change of fluorescence baseline on individual neurons. Spikes from two neurons with interval no more than ±2 frames were considered as occurring at the same time. A burst was counted when there were 50% neurons having calcium spikes at the same time in the network. For estimating network synchronization, pair-wise synchronization index of each two-neuron-pair was computed to get synchronization matrix based on either pairwise correlation coefficients or spikes instantaneous phase, which gave consistent results. Network synchronization index was acquired from the eigenvalues of the synchronization matrix. Higher synchronization index signified more synchronized activity in the neural network.

### EXAMPLE 1

Identification of Genetic Elements that Directly Convert Human Pluripotent Stem Cells (hPSCs) to GABAergic Neurons.

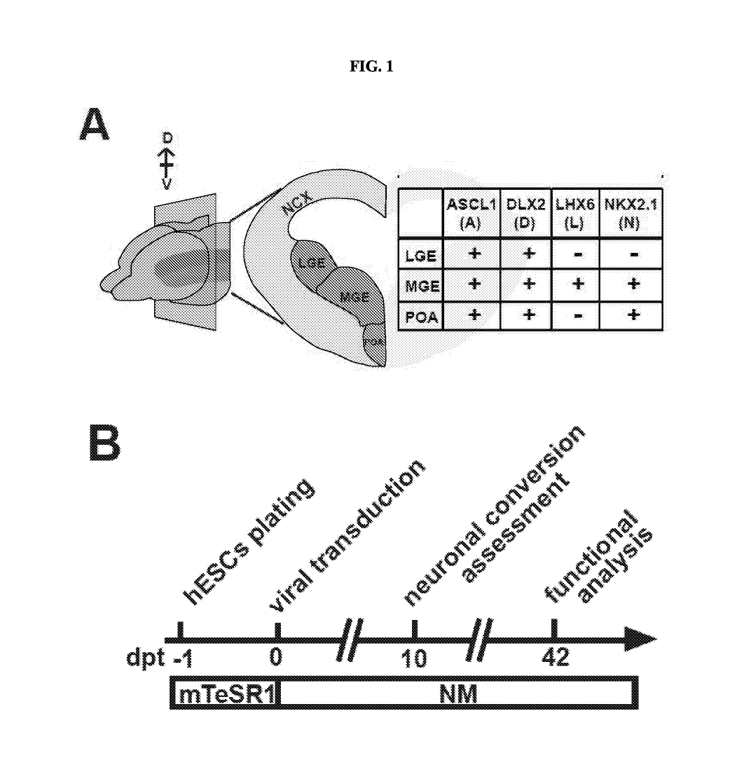

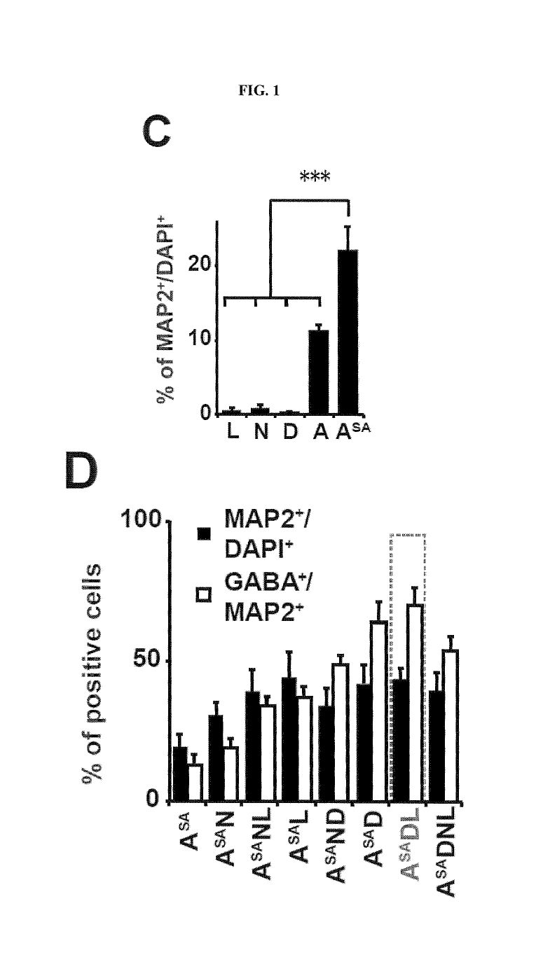

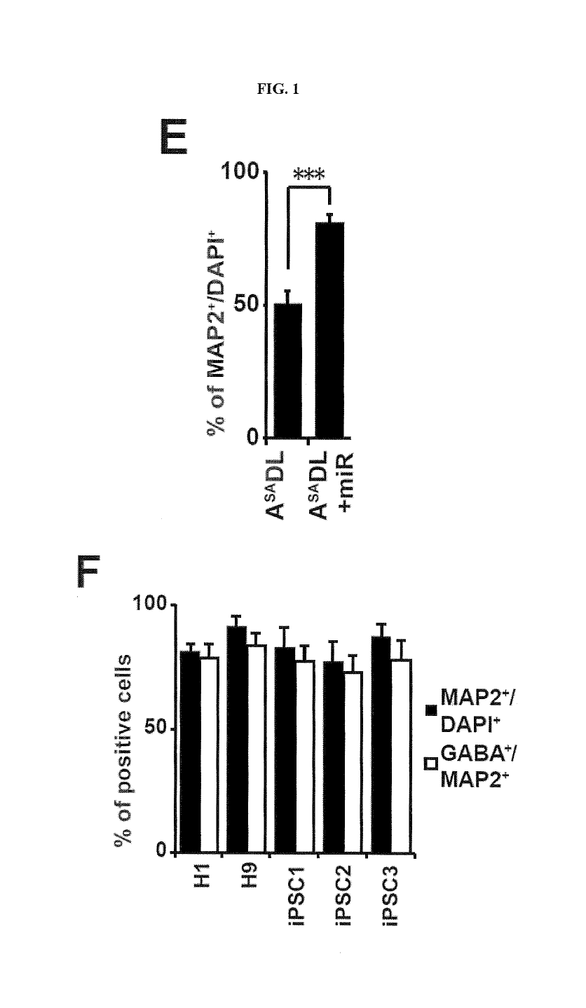

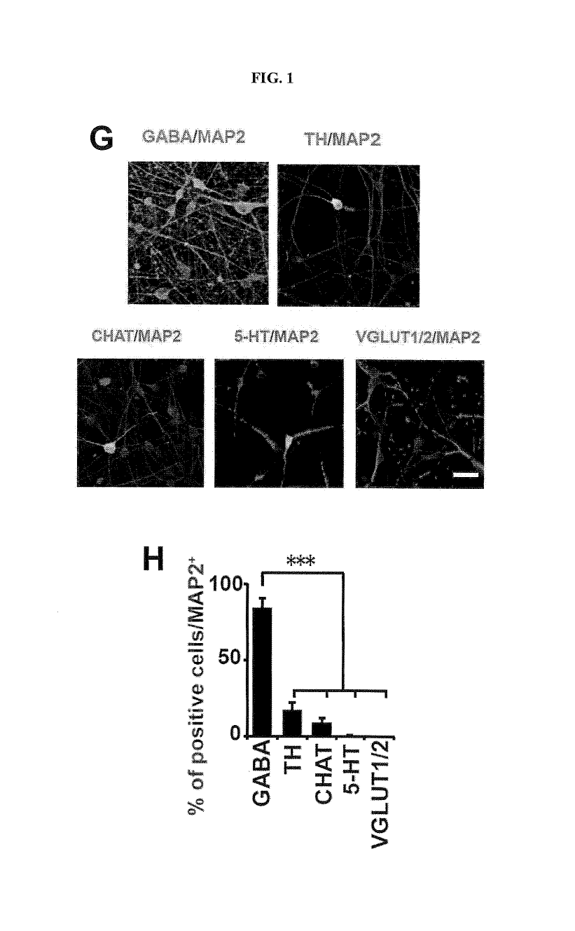

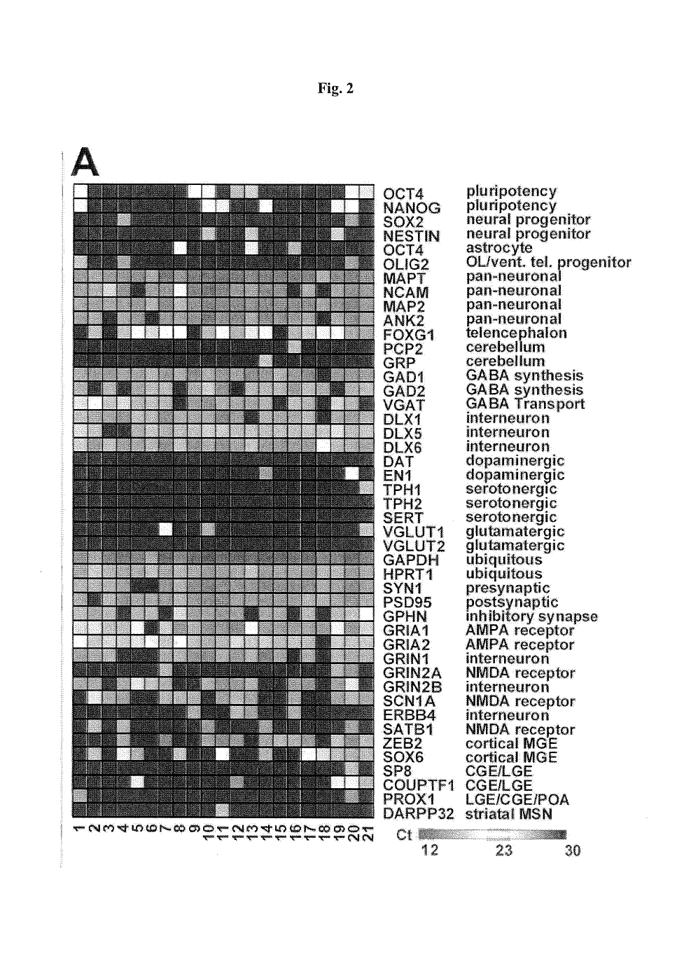

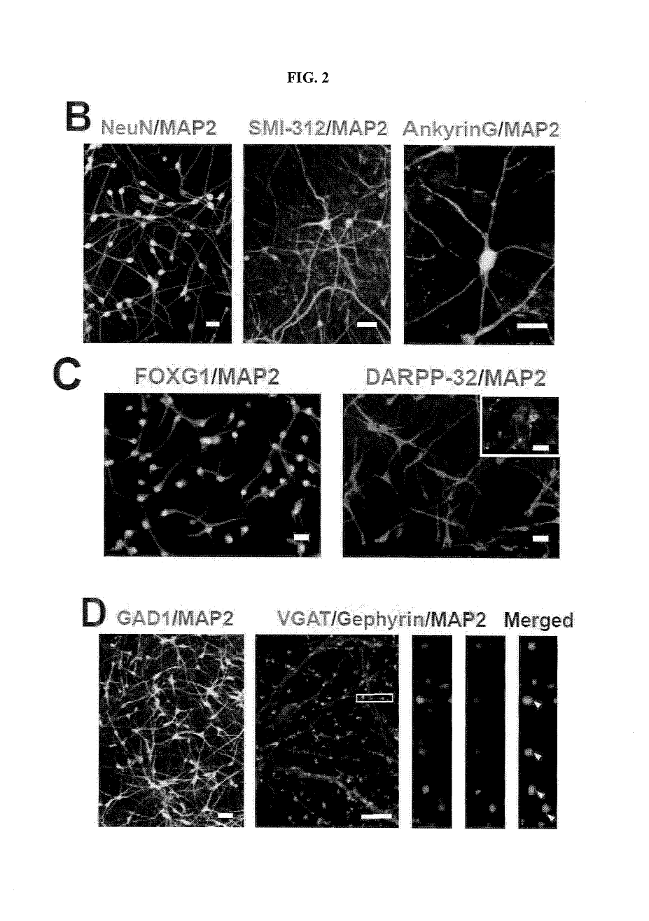

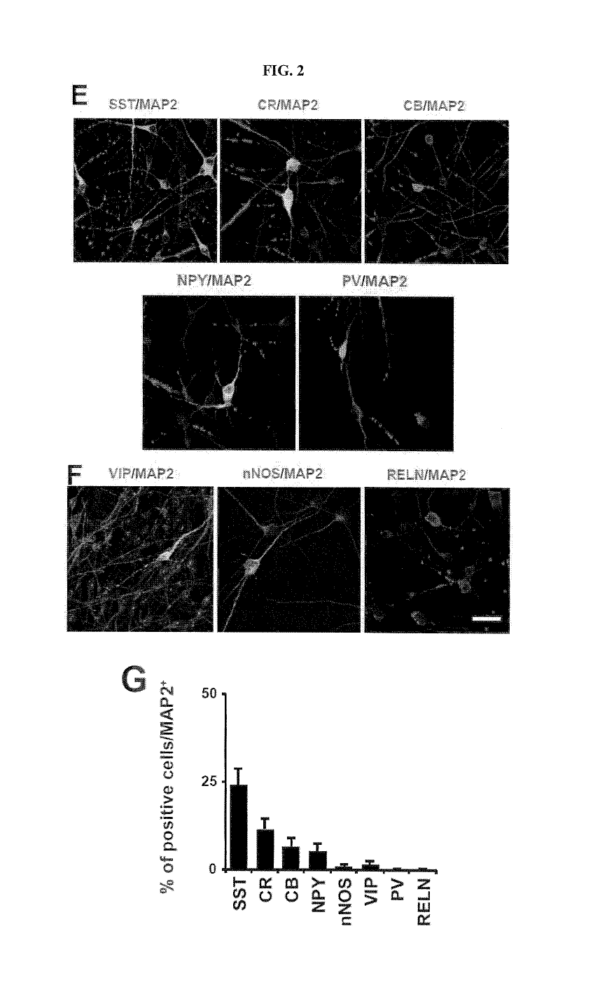

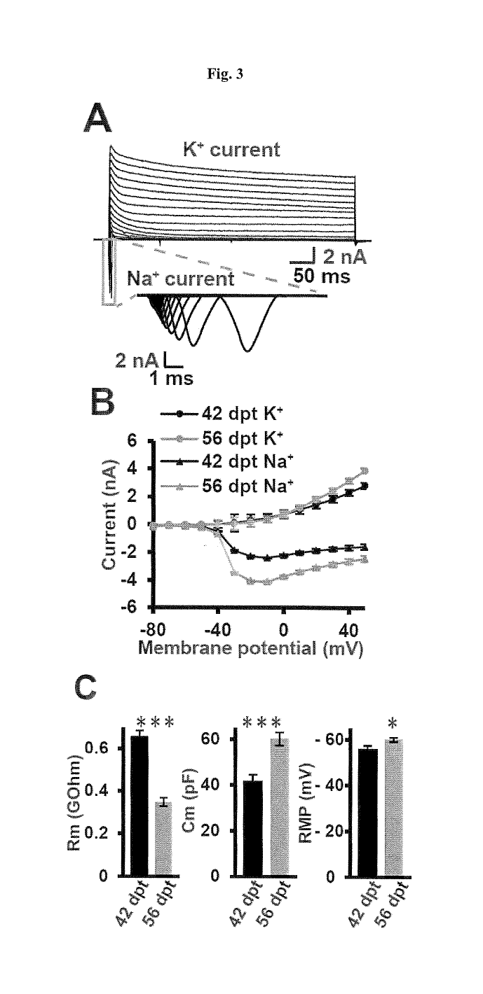

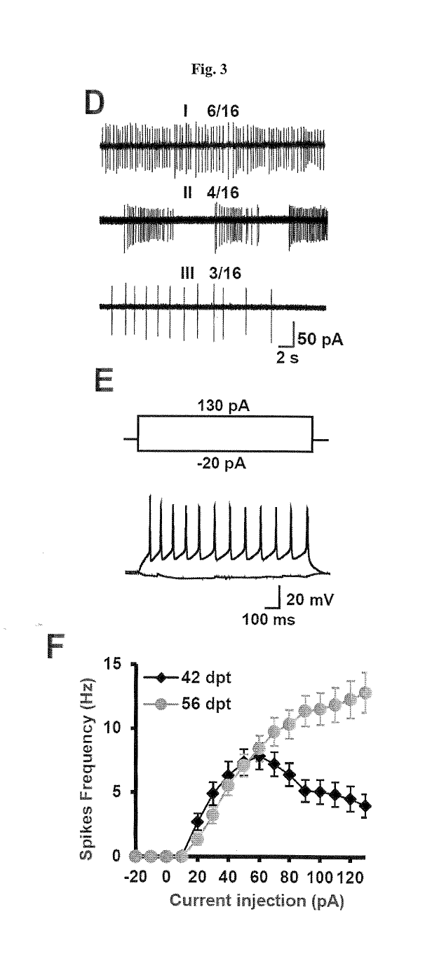

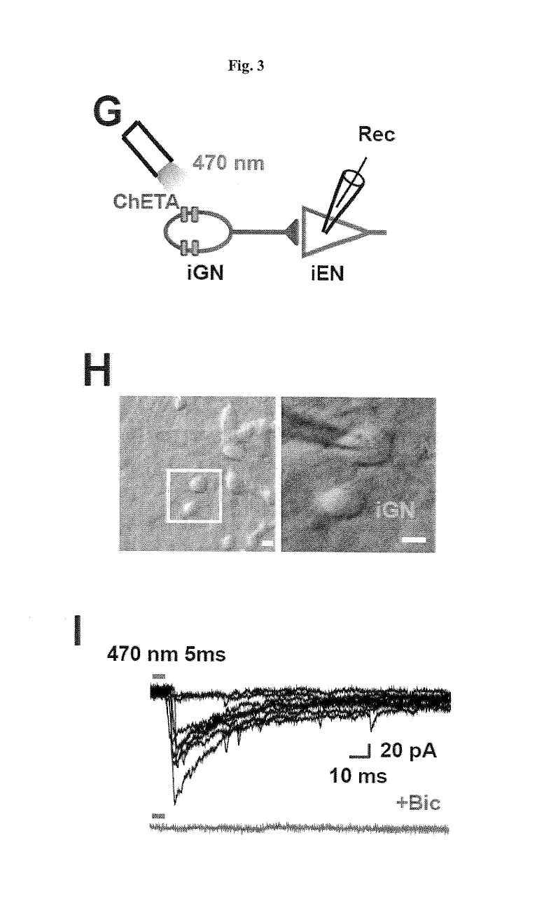

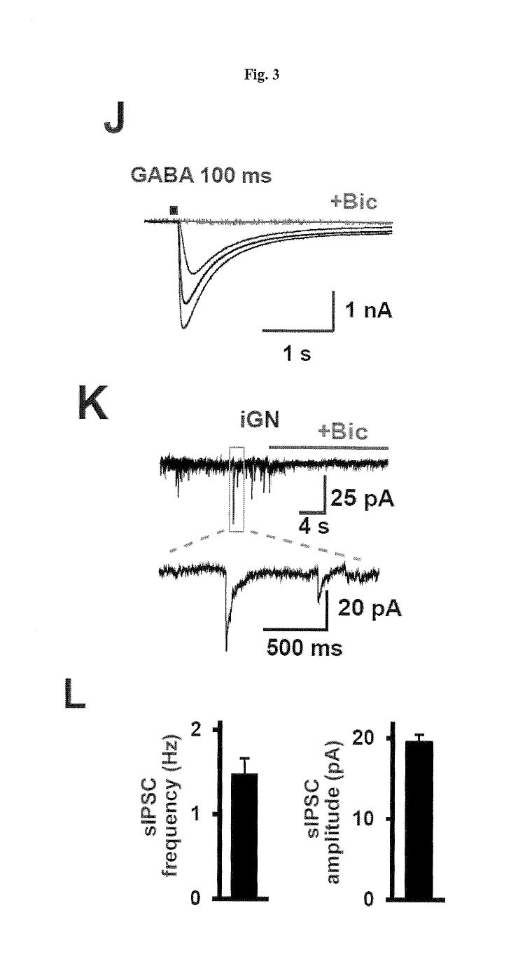

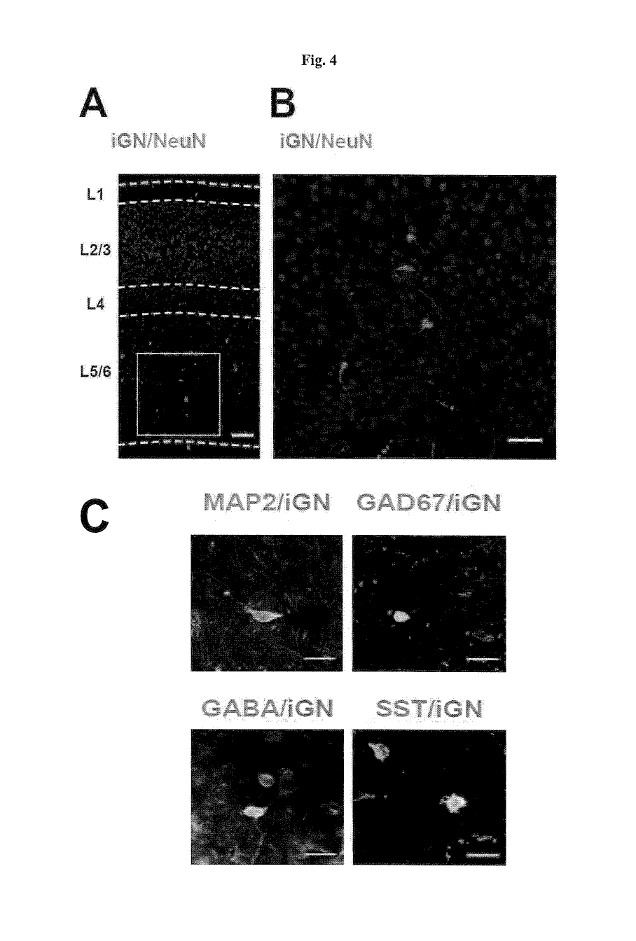

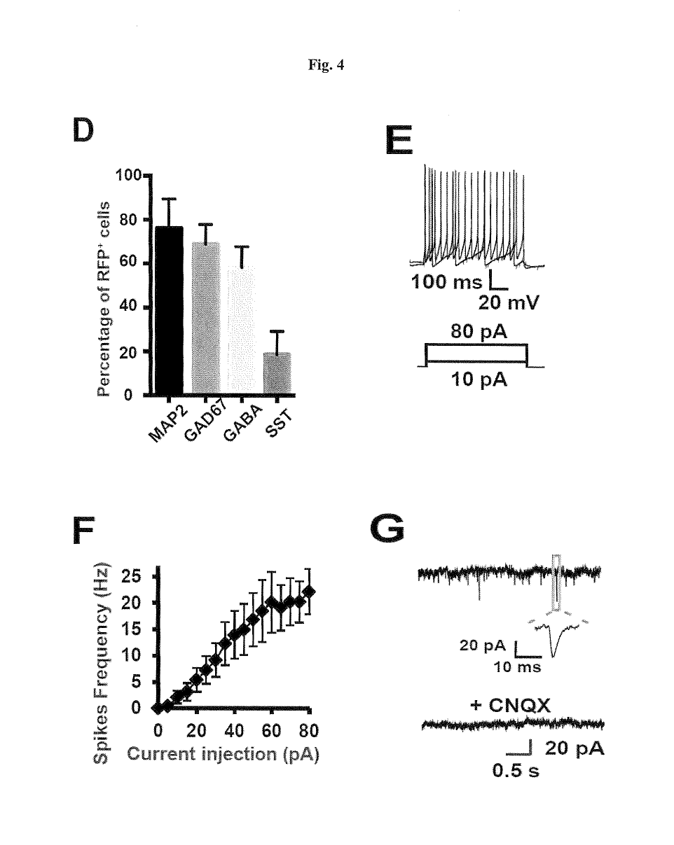

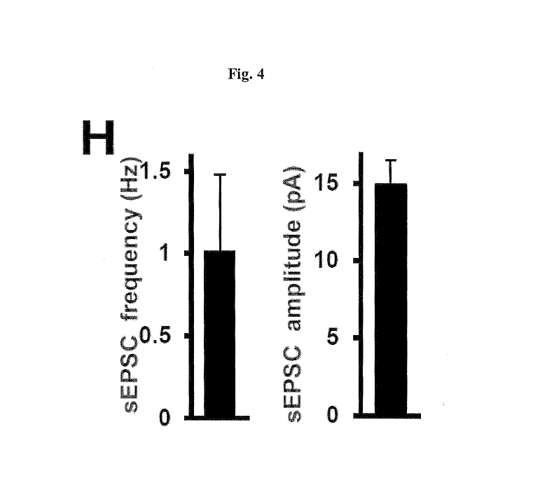

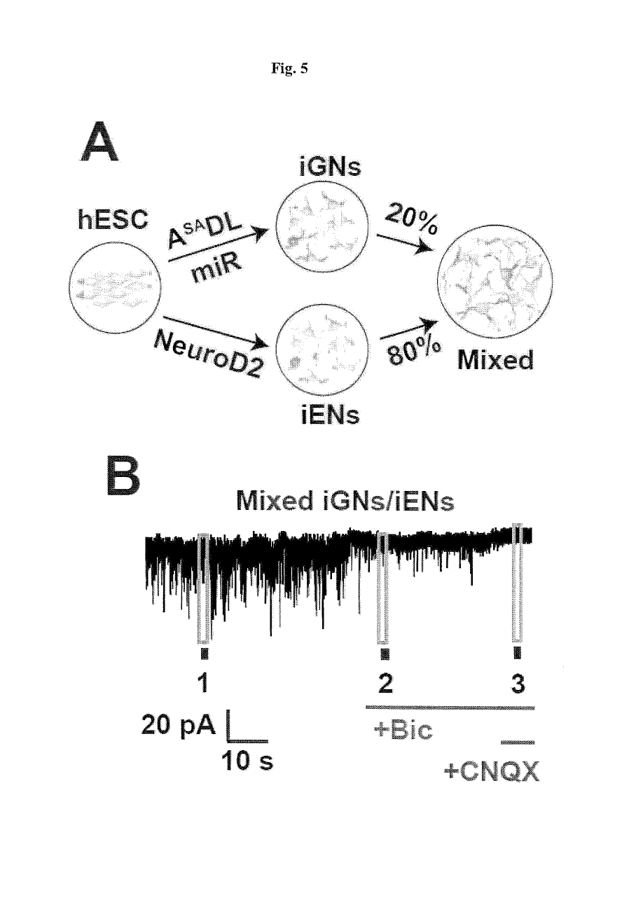

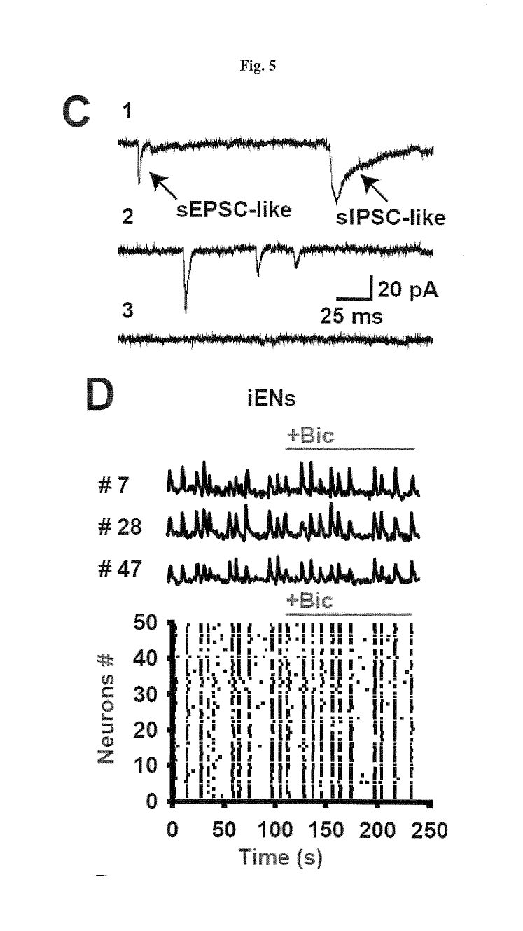

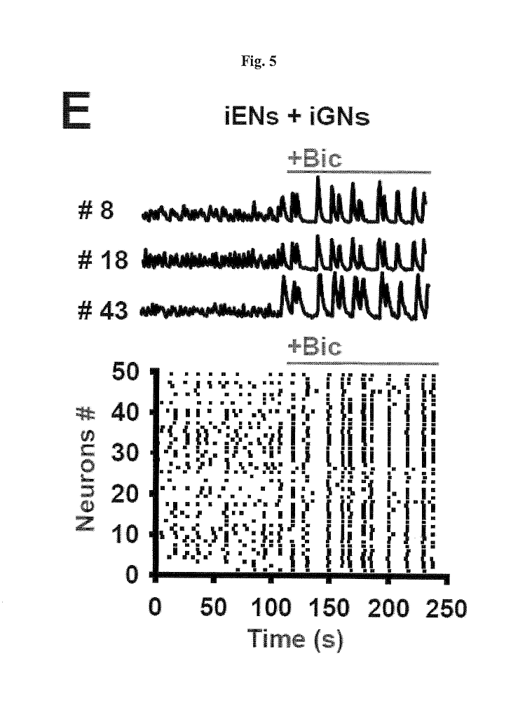

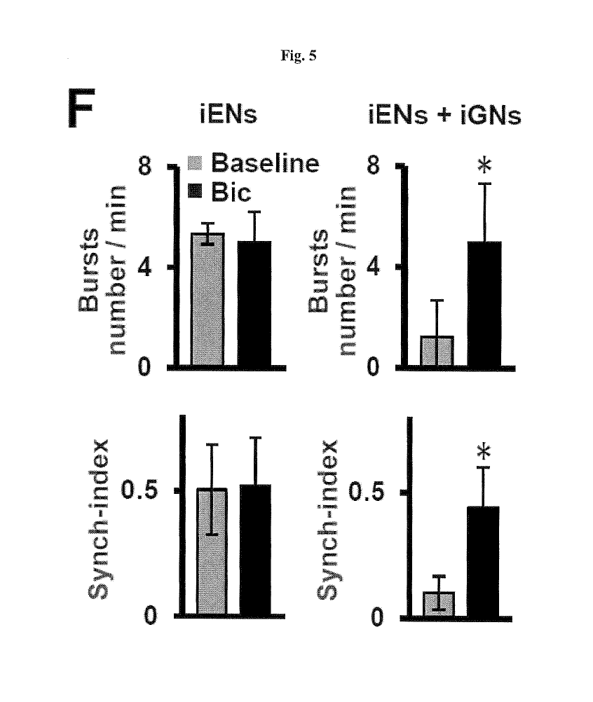

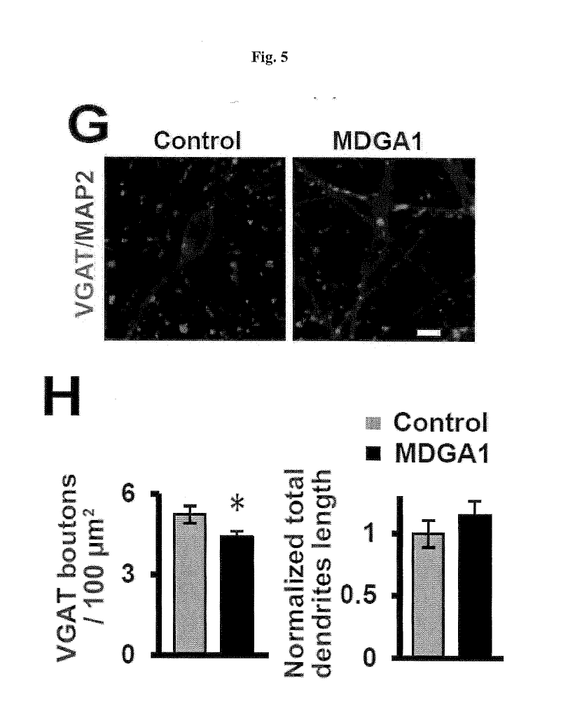

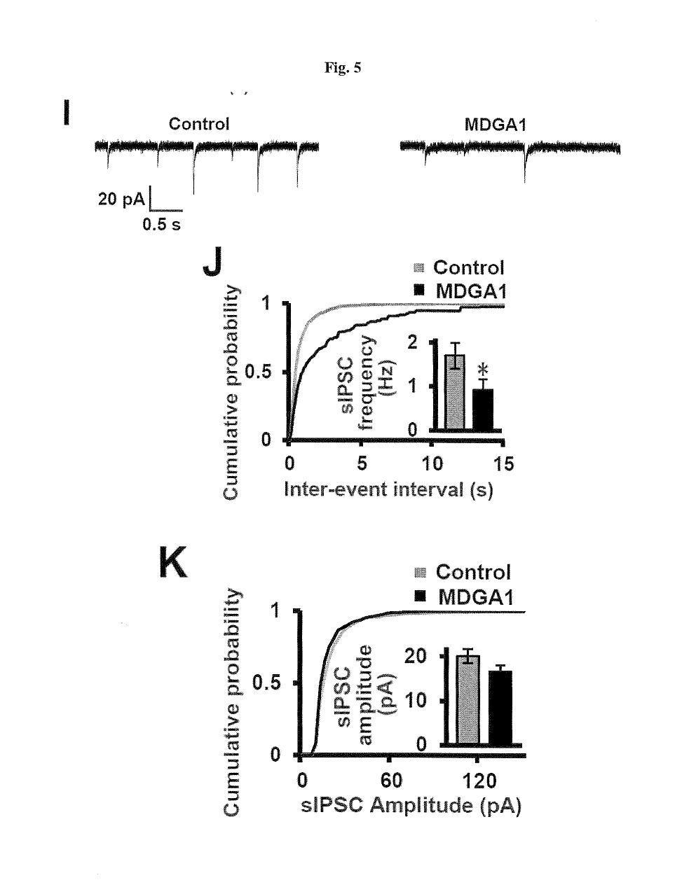

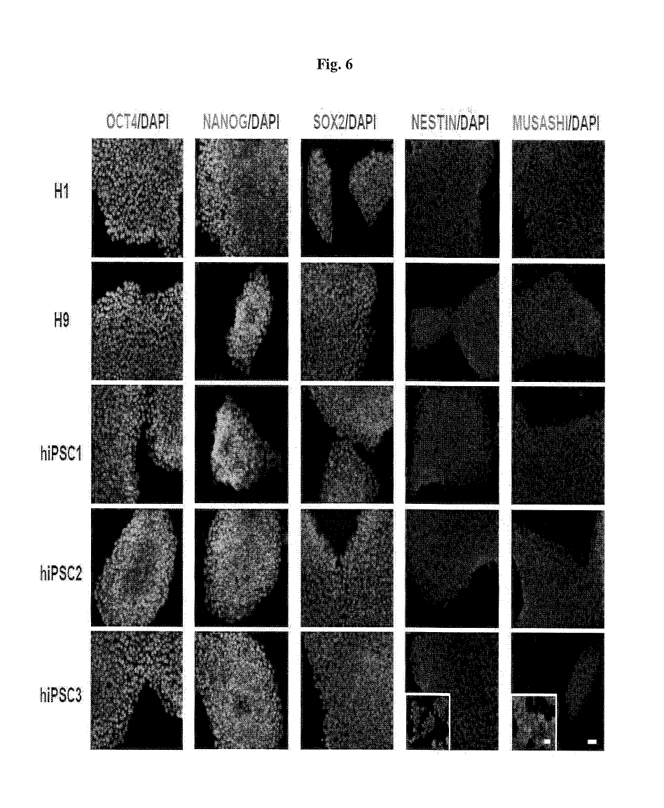

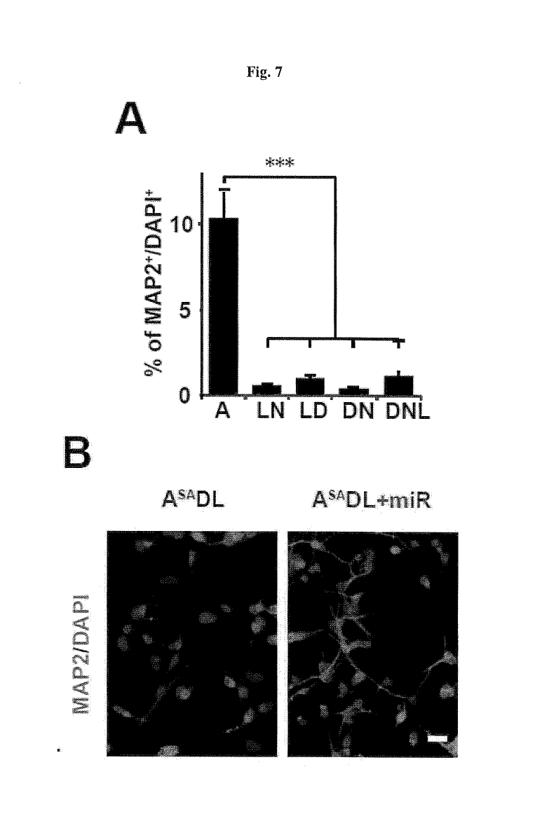

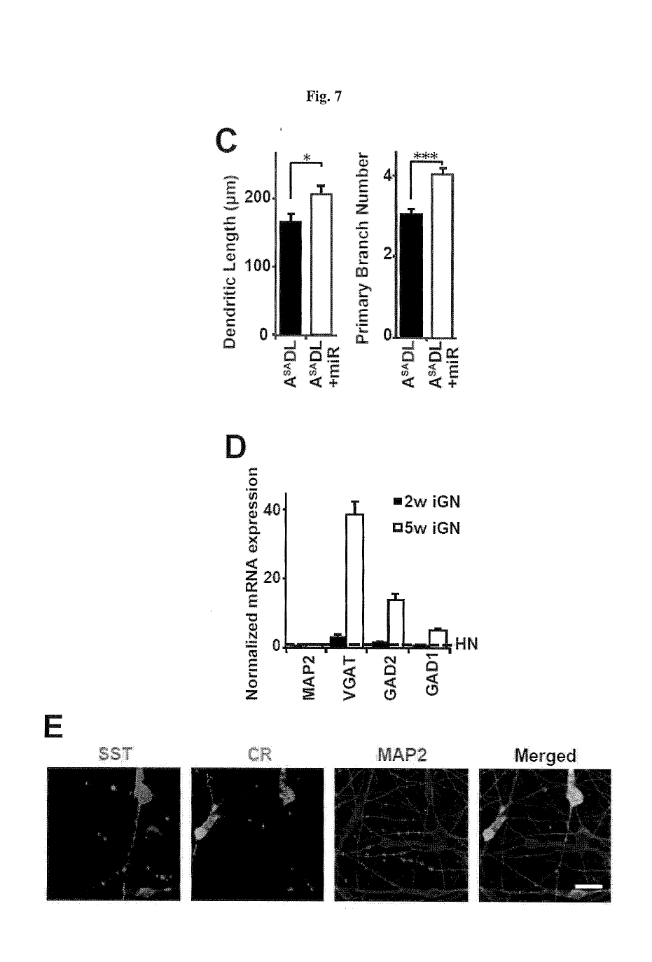

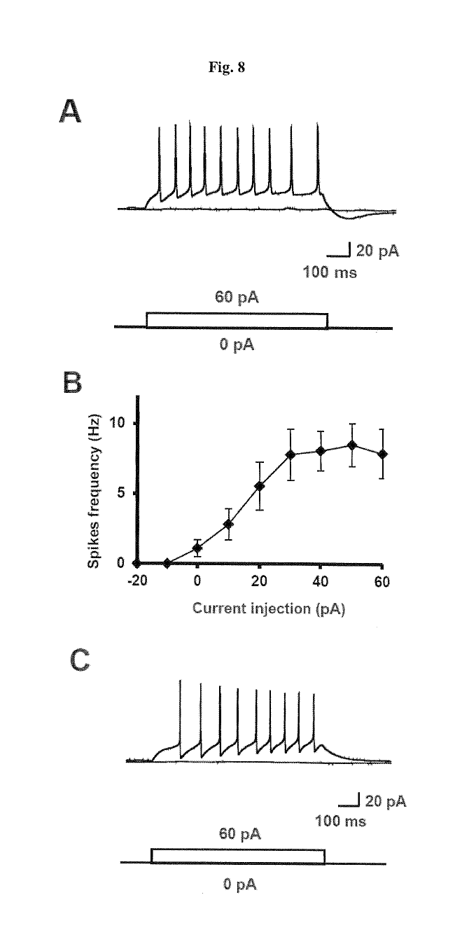

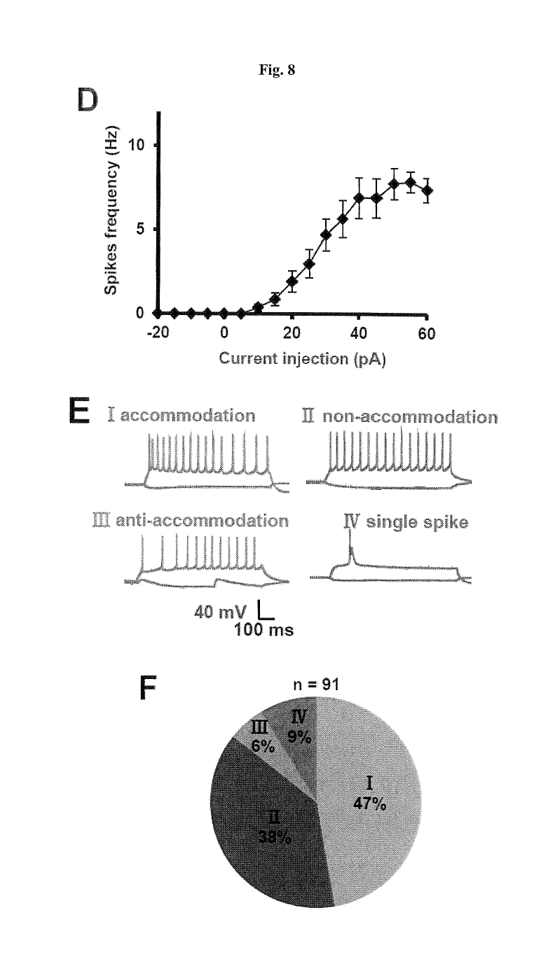

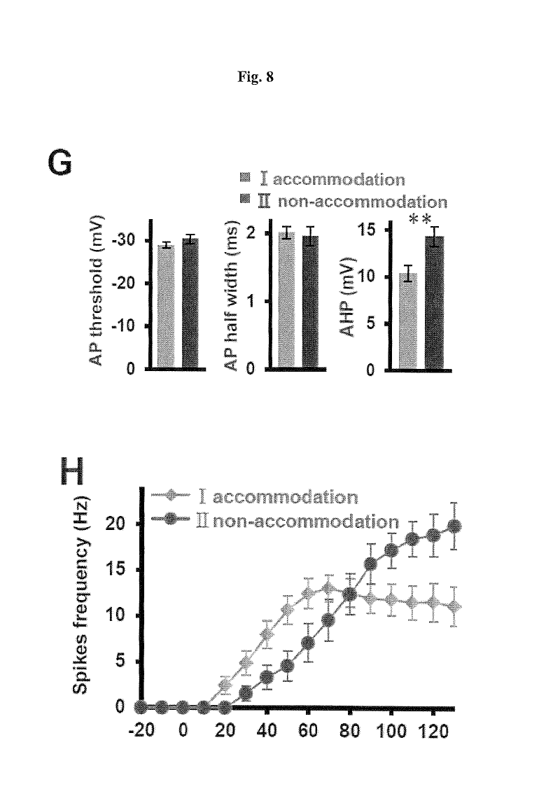

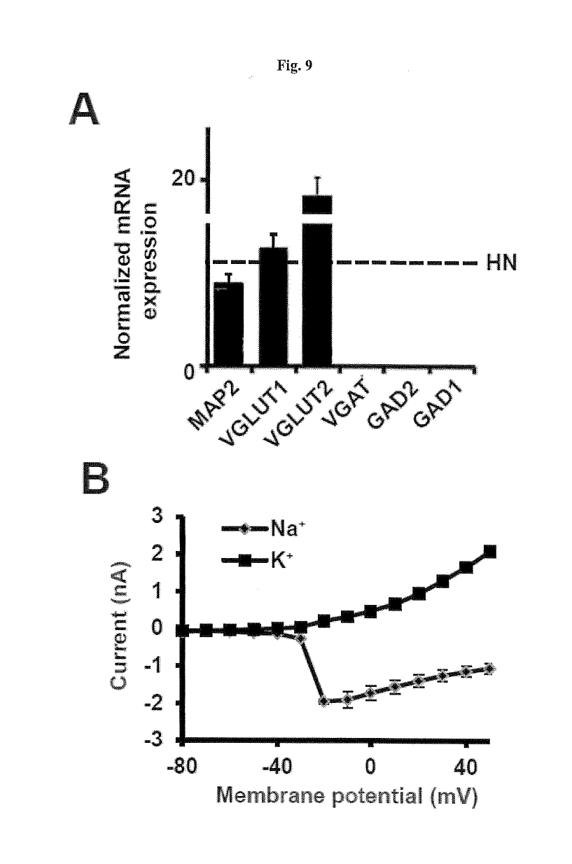

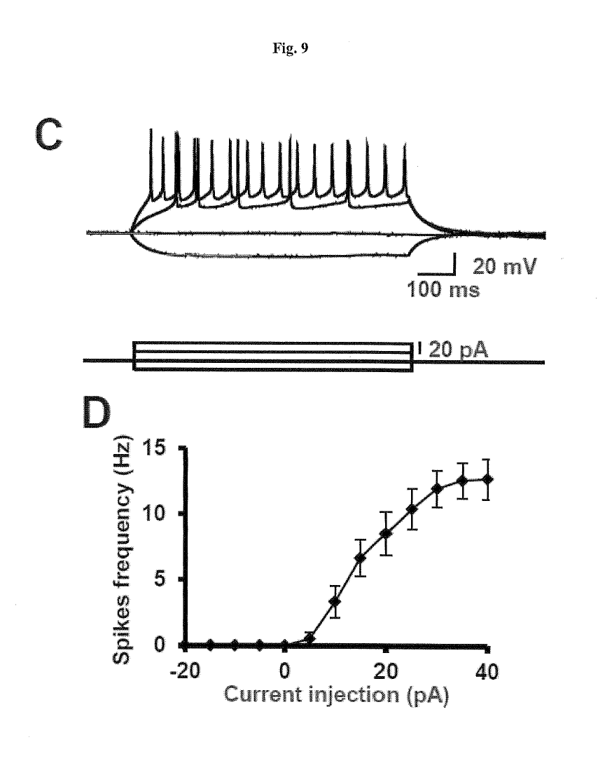

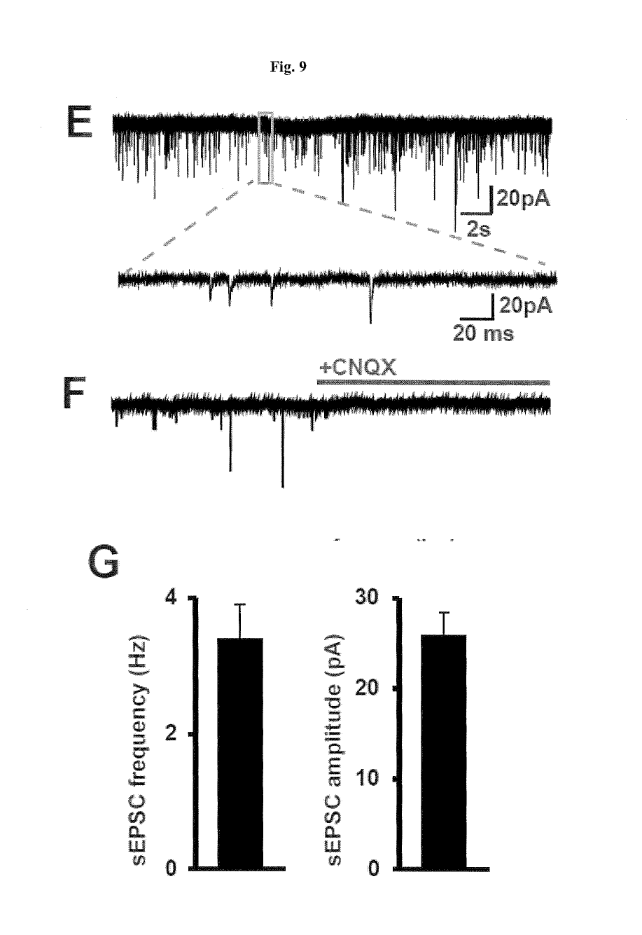

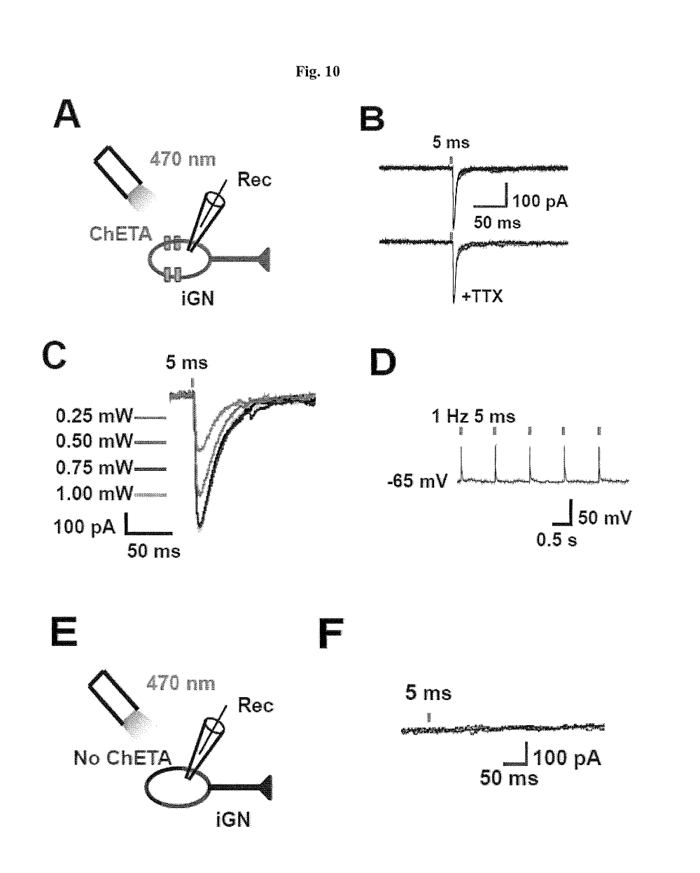

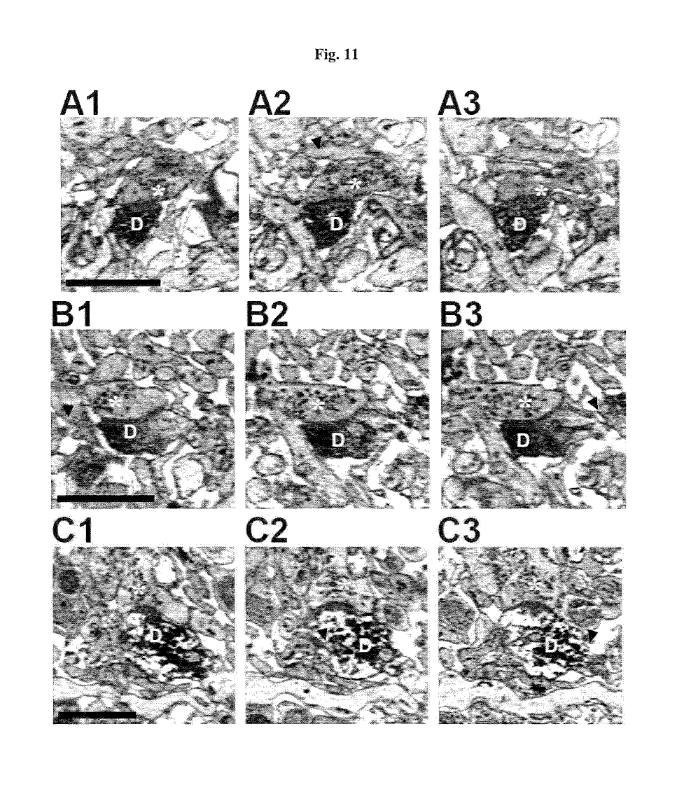

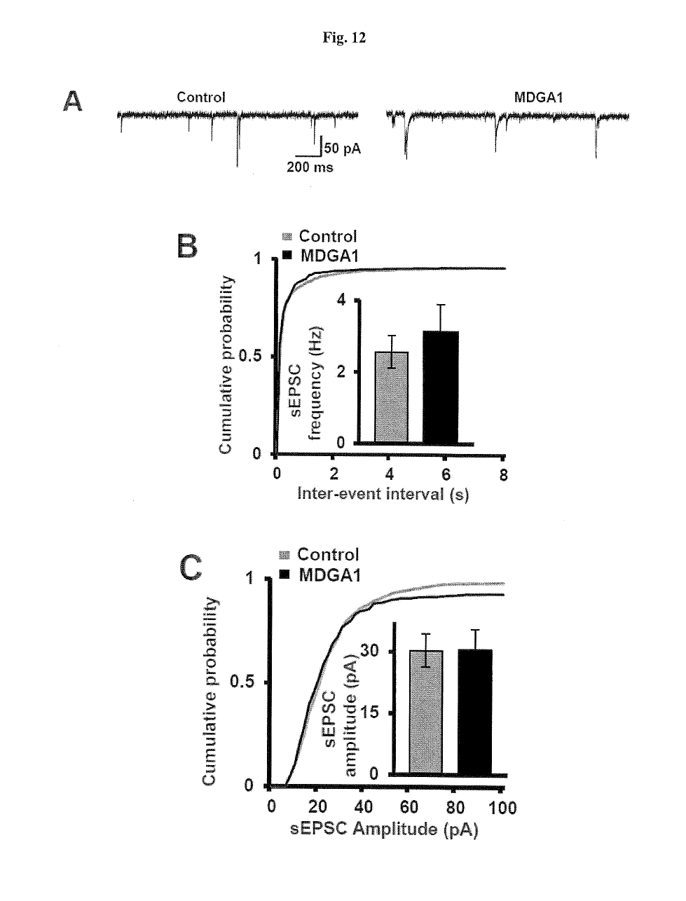

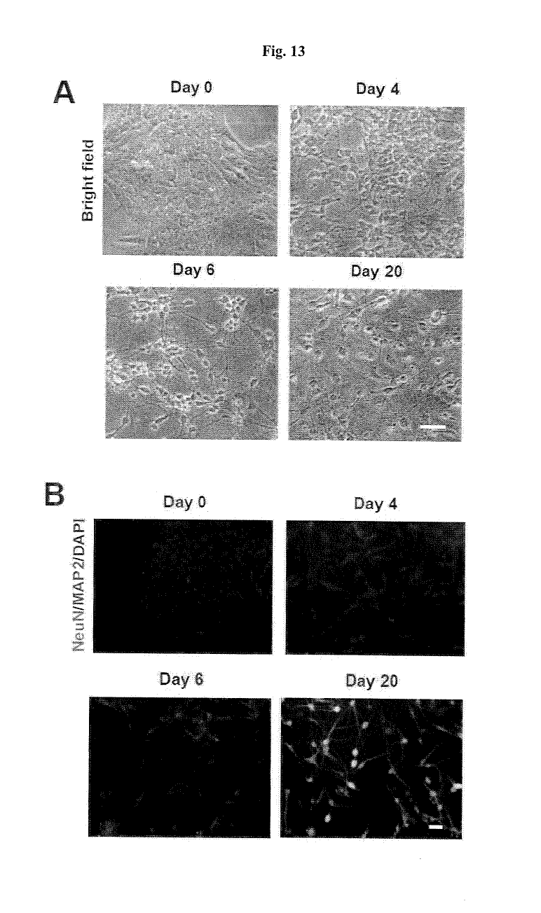

To identify genetic elements that can directly convert hESCs to GABAergic neurons, focus was placed on four transcription factors (TFs), ASCL1, DLX2, NKX2.1, and LHX6. These TFs are expressed in the medial ganglionic eminence (MGE), a major site of GABAergic neurogenesis, and are important for the differentiation and functional maturation of cortical interneurons (FIG. 1A). ASCL1 (achaete-scute complex-like homolog 1, also known as MASH 1) is a proneural bHLH factor that is widely expressed in the embryonic ventral brain, where it promotes the differentiation of neuronal progenitors into GABAergic intemeurons. DLX2 (distal-less homeobox 2) is a multifunctional TF that inhibits the differentiation of progenitors into glial cells and promotes their differentiation into GABAergic neurons. NKX2.1 (NK2 homeobox 1) controls the migration of telencephalic interneurons and also determines the subtype identity of interneuron progenitors within the MGE. LHX6 (LIM homeobox 6), which is a direct target of NKX2.1, is required for the specification of MGE precursors to parvalbumin (PV)- or somatostatin (SST)-expressing cortical interneurons.

Lentiviruses expressing each of the four TFs, infected hESCs (line H1) were generated, and their conversion to neuronal cells was assessed by staining the cells for the pan-neuronal marker MAP2 at 10 days post-transduction (dpt) (FIG. 1B). Initially hESCs expressed highly pluripotency markers such as OCT4, NANOG and SOX2, but did not express neural progenitor markers NESTIN and MUSASHI (FIG. 6). Intriguingly, the expression of ASCL1 (indicated as A) alone converted 11.3±0.8% of hESCs into MAP2-positive cells at 10 dpt, whereas the expression of the other TFs (DLX2 (D), LHX6 (L), and NKX2.1 (N)) failed to produce significant MAP2 signal (FIG. 1C). Similar results were obtained following immunohistochemical staining for another neuronal marker βIII tubulin (data not shown). All possible combinations of the D, N, and L TFs were subsequently tested, but neuronal conversion was not observed (FIG. 7A), indicating that ASCL1 plays an instructive role in converting hESCs to neurons, consistent with previous reports.

A phospho-mutant form of ASCL1 (in which 5 serine resides are substituted with alanine, denoted A^{SA}) is more potent than the wild-type ASCLI in ectopic neural induction in Xenopus embryos and in the trans-differentiation of human fibroblasts to neurons. Accordingly, A^{SA} was overexpressed in hESCs and it was found that it resulted in the production of approximately 2-fold more MAP2-positive neurons than A (FIG. 1C). Based on these results, A^{SA} was included as a neurogenic TF that is necessary to induce the differentiation of hESCs to GABAergic neurons.

Next, the possibility that the addition of other factors could enhance neuronal conversion and, more specifically, GABAergic neuronal conversion was investigated. To that end, hESCs were transduced with different pools of lentiviruses that contained A^{SA} as an obligatory factor together with various combinations of the other 3 TFs (D, N, and L) and quantified MAP2- and GABA- positive cells at 10 dpt (FIG. 1D). The majority of TF combinations significantly enhanced neuronal conversion over that produced by A^{SA} alone (FIG. 1D). Remarkably, a subset of them also dramatically increased the efficiency of GABAergic neuronal conversion, with A^{SA}DL producing the highest percentage of GABA and MAP2 double-positive cells (69.1±3.6%) (FIG. 1D). Taken together, these results show that D and L synergized with A^{SA} to preferentially produce GABAergic neurons from hESCs.

To further improve the overall conversion efficiency, miR-9/9*-124, which we increase neuronal conversion from non-neuronal cells were co-expressed, together with the A^{SA}, D, and L TFs. Strikingly, the addition of miR-9/9*-124 significantly increased the percentage of MAP2-positive cells from 50.3±4.7% to 81.3±3.1% (FIG. 1E) while maintaining the high ratio of GABA⁺/MAP² (FIG. 1F). Moreover, the expression of miR-9/9*-124 enhanced dendritic arborization (FIG. 7B), as demonstrated by an increase in the total dendritic length and number of primary branches of converted neurons (FIG. 7C). Importantly, a similar increase in neuronal conversion efficiency was observed across multiple hPSC lines, including two human ESCs (H1 and H9) and three different human iPSCs (FIG. 1F and FIG. 6). The neuronal conversion rate continued to rise, reaching more than 90% after 35 dpt across multiple cell lines (data not shown). The transduction of hESCs with miR-9/9*-124 alone failed to induce neuronal cells (data not shown). These results led to the conclusion that A^{SA}, D, L and miR-9/9*-124 (A^{SA}DL miR-9/9*-124) are optimal factors for the induction of GABAergic neurons from hPSCs.

To promote survival and functional maturation, cells that were transduced with A^{SA}DL+miR-9/9*-124 were co-cultured with rat glia Immunostaining at 42 dpt revealed that the majority of the induced neuronal cells were GABAergic (84.5±3.5%) (FIG. 1G and 1H). Small percentages of the converted neuronal cells expressed other neuronal markers, such as tyrosine hydroxylase (TH, a marker of dopaminergic neurons) and choline acetyltransferase (ChAT, a marker of cholinergic neurons) (FIG. 1G and 1H). Virtually none of the converted neuronal cells expressed markers for excitatory glutamatergic neurons, such as vesicular glutamate transporter 1 and 2 (VGLUT1/2), or the marker for serotonergic neurons 5-hydroxytryptamine (5-HT) (FIG. 1G and 1H). Taken together, these results demonstrate that the expression of A^{SA}DL+miR-9/9*-124 could robustly induce hPSCs to differentiate into a relatively homogenous population of GABA-producing neuronal cells (namely, iGNs).

Molecular Characterization of Human Induced GABAergic Neurons (iGNs)

To gain insight into the kinetics of how iGNs acquire an inhibitory neuronal fate, mRNA levels of key genes responsible for the GABAergic phenotype (VGAT, GAD1, and GAD2) were measured in iGNs using quantitative RT-PCR at 14 and 35 dpt. At 14 dpt, the iGNs expressed all three markers at levels similar to those observed in fetal human brains, which contained approximately 10-15% GABAergic interneurons (FIG. 7D). By 35 dpt, the expression of all three genes markedly increased (a 6- to 13-fold increase), indicating further maturation of the iGNs (FIG. 7D).

To further characterize the iGNs, multiplexed gene expression analysis of single iGNs was performed at 48-52 dpt (FIG. 2A and Table 1). A drastic erasure of pluripotency markers (OCT4 and NANOG) was observed with concurrent uniform expression of pan-neuronal markers (MAPT, NCAM, MAP2, and ANK2) (FIG. 2A). The expression of markers of neural progenitors (SOX2 and NESTIN), astrocytes (GFAP), and oligodendrocytes (OLIG2) was negligible (FIG. 2A). The iGNs expressed the telencephalic marker FOXG1 but not PCP2 and GRP, which are genes that are highly expressed in the cerebellum. Furthermore, consistent with the immunostaining data, markers of GABAergic interneurons (GAD1, GAD2, VGAT, DLX1, DLX5, and DLX6) were expressed in virtually all cells, whereas the expression of markers indicative of other neuronal lineages was largely absent (dopaminergic: DAT and EN1; serotonergic: TPH1, TPH2, and SERT; glutamatergic: VGLUT1 and VGLUT2) (FIG. 2A). The majority of the iGNs robustly expressed synaptic markers (SYN1, PSD95, and GPHN) as well as AMPA and NMDA receptors (GRIA1, GRIA2, GRIN1, GRIN2A, and GRIN2B). They also expressed genes that have been previously shown to be important for the function of interneurons, such as SCN1A, ERBB4 and SATB1(FIG. 2A). Finally, the majority of the iGNs expressed cortical, MGE-derived interneuronal markers such as ZEB2 and SOX6. However, the majority of the iGNs did not express SP8, COUPTF1, or PROX1; markers of interneurons with alternative birthplaces (CGE, LGE, and POA); or DARPP-32, a defining marker of striatal medium spiny neurons (MSNs). Collectively, these data strongly indicate that the iGNs constituted a nearly homogenous population of cortical GABAergic neurons.

To confirm and corroborate the mRNA expression data, immunostaining analyses of the iGNs were performed. The iGNs strongly expressed NeuN, a mature neuronal marker, the axonal marker SMI-312 and Ankyrin G, an axon initial segment marker (FIG. 2B). Not surprisingly, the majority of the iGNs stained positive for FOXG1, a forebrain marker, but were negative for DARPP-32, a marker of MSNs (FIG. 2C). Consistent with the GABAergic nature of the induced cells, GAD1 was readily detected in the iGNs (FIG. 2D). Importantly, it was observed that gephyrin-positive puncta were juxtaposed to VGAT-positive puncta along the dendrites of the iGNs, providing a morphological indication of GABAergic synapses (FIG. 2D).

Mature cortical interneurons can be divided into different subgroups based on their expression of neuropeptides and calcium-binding proteins, including SST, PV, calretinin (CR), calbindin (CB), neuropeptide Y (NPY), reelin (RELN), neuronal nitric oxide synthetase (nNOS), and vasoactive intestinal peptide (VIP). Immunostaining revealed that a subset of iGNs expressed SST (24.3%), CR (11.6%), CB (6.5%), and NPY (5.4%) (FIG. 2E and 2G). About 10% of MAP2⁺ cells were found to be double positive for SST and CR (FIG. 7E), while SST and CB double positive cells were not present. PV-positive signals could only be consistently observed after 70 dpt, albeit in a small number of the iGNs (<1%, 5 out of 576 neurons) (FIGS. 2E and 2G). Other GABAergic subtype markers were examined, including VIP (4%), nNOS (2%), and RELN (<1%) (FIG. 2F and 2G), and found them to be scarcely present. Taken together, the results indicate that the population of iGNs was enriched with SST-expressing GABAergic neurons.

Functional Characterization of Induced GABAergic Neurons

To explore whether the iGNs exhibited functional membrane properties similar to those of neurons, patch-clamp recordings of iGNs at 42 and 56 dpt were performed. In voltage-clamp mode, the iGNs showed fast, inactivating inward and outward currents, which likely correspond to the opening of voltage-dependent potassium (K⁺)- and sodium (Na⁺)-channels, respectively (FIG. 3A). The peaks of the voltage-gated Na⁺ and K⁴ currents increased significantly from 42 to 56 dpt (FIG. 3B). Consistent with this finding, a significant decrease in membrane resistance (Rm), a significant increase in membrane capacitance (Cm), and a more hyperpolarized resting membrane potential (RMP) was observed, indicating that iGNs were more mature at 56 dpt (FIG. 3C and Supplementary information, Table 2). These data show that the iGNs possessed the fundamental structural components required to function as neurons.

TABLE 2
Membrane resistance (Rm), membrane capacitance (Cm) and resting
membrane potential (RMP) of iGNs at 42 dpt and 56 dpt.

| | | | | | AP- | Spike | |
|---|---|---|---|---|---|---|---|
| Neurons | Cm | RM | Ra | RMP | threshold | AHP | Half AP |
| number | (pF) | (MOhm) | (MOhm) | (mV) | (mV) | (mV) | width (ms) |
| mean (42 | 41.41 ± 3.04 | 654.63 ± 31.42 | 14.49 ± 0.59 | −55.8 ± 1.58 | −42.05 ± 2.32 | 11.52 ± 1.02 | 2.44 ± 0.15 |
| dpt) | | | | | | | |
| mean (56 | 60.091 ± 3.37 | 348.33 ± 22.37 | 11.39 ± 0.61 | −60.06 ± 2.23 | −43.11 ± 1.58 | 11.98 ± 0.78 | 1.91 ± 0.09 |
| dpt) | | | | | | | |
| #1 | 30 | 900 | 13 | −43 | −48 | 21.5 | 1.91 |
| #2 | 25 | 600 | 18 | −48 | −41 | 10 | 2.11 |
| #3 | 25 | 900 | 16 | −43 | — | — | — |
| #4 | 40 | 900 | 11 | −50 | −50 | 5.5 | 2.23 |
| #5 | 25 | 750 | 15 | −43 | −41 | 13 | 1.90 |
| #6 | 32 | 650 | 13 | −54 | −46 | 22 | 1.58 |
| #7 | 30 | 600 | 10 | −58 | −47 | 0 | 2.46 |
| #8 | 29 | 900 | 12 | −43 | −40 | 14 | 3.29 |
| #9 | 68 | 530 | 16 | −68 | −40 | 16 | 3.03 |
| #10 | 50 | 800 | 12 | −68 | — | — | — |
| #11 | 50 | 900 | 18 | −61 | −44 | 4 | 1.99 |
| #12 | 42 | 430 | 20 | −65 | −38 | 17 | 3.47 |
| #13 | 33 | 350 | 14 | −43 | −46 | 9 | 1.53 |
| #14 | 23 | 700 | 14 | −48 | — | — | — |
| #15 | 27 | 600 | 6 | −75 | −38 | 10 | 1.95 |
| #16 | 53 | 450 | 20 | −73 | −38 | 14 | 2.09 |
| #17 | 57 | 900 | 8 | −63 | −42 | 15 | 1.54 |
| #18 | 30 | 900 | 10 | −51 | — | — | — |
| #19 | 46 | 450 | 9 | −71 | −38 | 21 | 2.85 |
| #20 | 36 | 700 | 10 | −68 | −34 | 17 | 2.62 |
| #21 | 44 | 450 | 14 | −53 | −38 | 14 | 2.71 |
| #22 | 28 | 600 | 12 | −48 | −53 | 14 | 1.42 |
| #23 | 37 | 450 | 10 | −73 | −40 | 16 | 2.40 |
| #24 | 22 | 800 | 4 | −48 | −37 | 18 | 2.20 |
| #25 | 57 | 550 | 11 | −70 | −39 | 4 | 2.30 |
| #26 | 55 | 450 | 18 | −60 | −35 | 10 | 2.36 |
| #27 | 37 | 800 | 15 | −57 | −37 | 11 | 2.83 |
| #28 | 35 | 450 | 12 | −63 | −40 | 10 | 3.58 |
| #29 | 28 | 800 | 15 | −48 | −45 | 11 | 3.53 |
| #30 | 39 | 600 | 10 | −56 | −40 | 8 | 3.02 |
| #31 | 65 | 450 | 13 | −64 | −43 | 16 | 2.93 |
| #32 | 42 | 550 | 9 | −55 | −38 | 11 | 2.41 |
| #33 | 25 | 900 | 15 | −41 | — | — | — |
| #34 | 38 | 550 | 19 | −58 | −43 | 0 | 1.91 |
| #35 | 135 | 100 | 15 | −58 | −35 | 14 | 2.79 |
| #36 | 55 | 780 | 19 | −53 | −39 | 7 | 3.42 |
| #37 | 40 | 800 | 15 | −53 | −45 | 8 | 2.57 |
| #38 | 45 | 900 | 17 | −43 | −47 | 13 | 1.86 |
| #39 | 30 | 500 | 16 | −43 | −55 | 5 | 2.29 |
| #40 | 35 | 900 | 15 | −48 | −49 | 11 | 2.15 |
| #41 | 55 | 550 | 14 | −60 | −45 | 5 | 2.61 |
| #42 | 50 | 350 | 15 | −71 | −38 | 20 | 1.66 |
| #43 | 62 | 450 | 18 | −63 | −42 | 4 | 1.55 |
| #44 | 65 | 350 | 10 | −58 | −42 | 9 | 1.73 |
| #45 | 48 | 490 | 10 | −55 | −36 | 20 | 2.45 |
| #46 | 50 | 450 | 12 | −63 | −37 | 19 | 1.74 |
| #47 | 35 | 600 | 13 | −58 | — | — | — |
| #48 | 53 | 370 | 12 | −43 | −51 | 12 | 1.85 |
| #49 | 52 | 250 | 10 | −49 | −45 | 16 | 1.60 |
| #50 | 37 | 240 | 19 | −65 | −33 | 18 | 2.44 |
| #51 | 35 | 500 | 12 | −48 | −48 | 8 | 1.77 |
| #52 | 53 | 180 | 10 | −71 | −45 | 13 | 1.48 |
| #53 | 65 | 300 | 11 | −73 | −41 | 13 | 1.45 |
| #54 | 55 | 500 | 17 | −63 | −43 | 6 | 2.95 |
| #55 | 27 | 260 | 3 | −63 | −36 | 15 | 2.58 |
| #56 | 88 | 200 | 13 | −62 | −44 | 7 | 1.40 |
| #57 | 47 | 150 | 10 | −71 | — | — | — |
| #58 | 57 | 200 | 20 | −58 | −37 | 5 | 2.86 |
| #59 | 46 | 350 | 8 | −54 | −35 | 13 | 2.63 |
| #60 | 110 | 280 | 11 | −68 | −46 | 14 | 1.58 |
| #61 | 95 | 300 | 12 | −73 | — | — | — |
| #62 | 38 | 450 | 9 | −56 | −39 | 21 | 1.89 |
| #63 | 70 | 420 | 15 | −54 | −42 | 5 | 1.81 |
| #64 | 65 | 220 | 15 | −61 | −35 | 13 | 3.76 |
| #65 | 110 | 400 | 8 | −65 | −45 | 10 | 2.15 |
| #66 | 57 | 280 | 14 | −65 | −40 | 13 | 2.25 |
| #67 | 55 | 900 | 8 | −68 | −39 | 6 | 1.48 |
| #68 | 57 | 450 | 6 | −64 | −46 | 15 | 1.93 |
| #69 | 60 | 450 | 15 | −68 | −41 | 15 | 2.36 |
| #70 | 86 | 180 | 15 | −61 | −43 | 19 | 2.96 |
| #71 | 60 | 220 | 15 | −48 | −45 | 3 | 1.59 |
| #72 | 60 | 500 | 8 | −58 | −52 | 13 | 1.41 |
| #73 | 80 | 400 | 20 | −63 | −50 | 6 | 1.26 |
| #74 | 56 | 320 | 11 | −63 | −51 | 11 | 1.36 |
| #75 | 75 | 490 | 17 | −58 | −43 | 9 | 1.44 |
| #76 | 90 | 200 | 15 | −63 | −45 | 4 | 1.87 |
| #77 | 40 | 140 | 10 | −63 | −48 | 12 | 1.25 |
| #78 | 40 | 470 | 8 | −71 | −47 | 14 | 1.60 |
| #79 | 60 | 450 | 11 | −54 | — | — | — |
| #80 | 85 | 200 | 8 | −54 | −47 | 5 | 2.08 |
| #81 | 51 | 450 | 10 | −59 | −48 | 19 | 1.37 |
| #82 | 45 | 270 | 11 | −55 | −44 | 10 | 1.57 |
| #83 | 110 | 140 | 8 | −65 | −45 | 17 | 1.26 |
| #84 | 58 | 240 | 8 | −58 | −49 | 8 | 1.36 |
| #85 | 75 | 160 | 11 | −55 | −45 | 7 | 2.64 |
| #86 | 55 | 180 | 9 | −65 | −55 | 13 | 1.57 |
| #87 | 47 | 450 | 9 | −54 | −40 | 13 | 2.23 |
| #88 | 45 | 480 | 6 | −63 | −44 | 19 | 1.36 |
| #89 | 41 | 480 | 8 | −53 | −40 | 12 | 1.59 |
| #90 | 98 | 240 | 7 | −48 | −43 | 6 | 1.72 |
| #91 | 33 | 600 | 7 | −55 | −40 | 13 | 1.65 |
| #92 | 35 | 240 | 14 | −53 | −41 | 13 | 1.61 |
| #93 | 58 | 400 | 10 | −70 | −45 | 4 | 2.22 |
| #94 | 90 | 400 | 11 | −53 | −45 | 5 | 1.69 |
| #95 | 30 | 170 | 12 | −51 | −42 | 10 | 2.29 |
| #96 | — | — | — | — | −47 | 20 | 2.18 |
| #97 | — | — | — | — | −45 | 20 | 2.24 |
| #98 | — | — | — | — | −37 | 13 | 2.74 |
| #99 | — | — | — | — | −36 | 19 | 2.04 |

Underlined #: 42 dpt iGNs;
Non-underlined #: 56 dpt iGNs

Next, both spontaneous and elicited action potentials (APs) were recorded from iGNs in cell-attached mode (FIG. 3D) and in current-clamp mode (FIG. 3E and F), respectively. Upon current injection, iGNs at 56 dpt showed enhanced AP firing (FIG. 3F). Electrophysiological recordings were performed from iGNs derived from additional cell lines and observed similar results (FIG. 8A-D). The iGNs were further characterized based on their AP firing patterns (FIG. 8E). When the iGNs were grouped based on their AP firing, the majority displayed an accommodating pattern (type I, 47%) or non-accommodating pattern (type II, 38%), whereas anti-accommodating (type III, 6%) and single AP firing (type IV, 9%) patterns were also observed in small subsets of the iGNs (FIG. 8F). These observations are consistent with previous studies that showed that the AP firing of the majority of SST-positive neurons in the neocortex typically displays an accommodating pattern. Fast-spiking or bursts of APs were not detected in the recordings from iGNs (data not shown). However, it was found that the non-accommodating iGNs exhibited a greater firing frequency and larger AHP compared to the accommodating iGNs (FIG. 8G and 8H). Furthermore, small current injections (<70 pA) induced more frequent AP firing in type I cells than in type II cells, but the type II cells fired more APs in response to larger current injections (>100 pA) (FIG. 8H). Taken together, these data indicate that the iGNs displayed different types of AP firing patterns, similar to cortical interneurons.

Next, to further confirm that the iGNs expressed the functional presynaptic machinery needed to release GABA and induce inhibitory postsynaptic responses, iGNs were co-cultured with iENs that were generated via a protocol similar to that used in a previous study (FIG. 9). First, ChETA, an engineered channelrhodopsin variant was expressed, only in iGNs; then, the ChETA-expressing iGNs were stimulated with an optical fiber coupled to a high-intensity blue light-emitting diode (LED) (FIG. 3G and 3H). Blue LED illumination (470 nm) induced ChETA-mediated inward currents and AP firing in iGNs, whereas the same photostimulation did not induce such currents in neurons that did not express ChETA, demonstrating that the iGNs could be selectively activated using this approach (FIG. 10). Under these conditions, short pulses of photostimulation that activated the ChETA-expressing iGNs induced postsynaptic responses in iENs (FIG. 3I). The recorded postsynaptic currents showed short synaptic delays, indicating they were induced monosynaptically (FIG. 3I). Moreover, these light-evoked synapse responses were completely inhibited by bicuculline, a GABA_{A} receptor antagonist (FIG. 3I). These results indicate that the activation of iGNs could generate inhibitory postsynaptic responses in co-cultured iENs.

Whether the iGNs exhibited functional postsynaptic mechanisms, which would enable synaptic transmission in situ was further investigated. First, exogenous application of GABA (1 mM, 100 ms) triggered inhibitory postsynaptic currents (IPSCs) in iGNs (FIG. 3J). These IPSCs were also completely blocked by bicuculline, indicating that the iGNs expressed functional GABA_{A} receptors (FIG. 3J). Second, at 42 to 56 dpt, the majority of the iGNs showed spontaneous inhibitory postsynaptic currents (sIPSCs) at a mean frequency of 1.47 Hz (1.47±0.18 Hz, n=61) (FIG. 3K and 3L). The mean sIPSC amplitude was 19.5±1.38 pA, which was comparable to that found in cultured rodent cortical neurons (FIG. 3K and 3L)(data not shown). These sIPSCs were completely inhibited by bicuculline (FIG. 3K), indicating that the human iGNs possessed functional postsynaptic machinery and received inhibitory synaptic inputs.

Functional Maturation and Synaptic Integration of Human iGNs In Vivo

To test whether the iGNs were able to undergo synaptic maturation and functional integration in vivo, RFP-expressing iGNs were stereotaxically transplanted at 8 dpt into cortices of P1 neonatal immunodeficient NOD SCID mice. Two months later, NeuN expressing iGNs were dispersed in mouse cortex, mostly in layer 5/6 (FIG. 4A and B). Quantification revealed that majority of the iGNs was positive for neuronal marker MAP2 and GABAergic markers GAD67 and GABA, indicating successful establishment of GABAergic neuronal identity (FIG. 4C and 4D). Approximately 20% of transplanted iGNs expressed SST. PV-positive human cells could be observed as well, however, their signal was much weaker than the endogenous mouse PV-positive neurons (data not shown). These results are consistent with our quantifications with cultured iGNs, and indicated that our protocol primarily yielded SST⁺ interneurons.

To determine whether transplanted iGNs develop into functional neurons and integrate into host neural circuitry, whole-cell patch-clamp recordings were used in acute cortical slices obtained from transplanted mice. Grafted iGNs, identified by RFP expression, displayed repetitive AP firings (FIG. 4E and 4F). Furthermore, spontaneous excitatory postsynaptic currents at −70 mV in voltage-clamp mode could be measured from transplanted iGNs in acute cortical slices and these synaptic currents were abolished in the presence of CNQX, an AMPA/kainate-type glutamate receptor antagonist (FIG. 4G and 4H). To further confirm the functional synapse formation between host and transplanted iGNs, ultrastructural analysis was performed using serial block face-scanning electron microscopy (SBF-SEM). Examination of cortical area in brain slices immunostained with diaminobenzidine (DAB) for RFP showed synaptic connections onto grafted iGNs (FIG. 11). These results demonstrate that the transplanted human iGNs are electrically excitable and are able to integrate into host neural circuitry by forming functional synapses.

Potential Use of iGNs in Large-Population Calcium Imaging and Interneuron-Specific Mechanistic Studies

To explore the potential use of the iGNs to assess cell type-specific drug effects or to model human disease states, iGNs were tested to see if they could form functional synaptic connections with other excitatory glutamatergic neurons. iGNs (20%) were co-cultured with iENs (80%), which mimics the proportions found in mammalian cortical networks, and measured spontaneous PSCs from the iGNs (FIG. 5A). Intriguingly, when neurons were clamped at −70 mV, two distinct patterns of PSCs could be measured (FIG. 5B and 5C). Treatment with bicuculline eliminated the slow-decay PSCs that resembled sIPSCs, and additional treatment with CNQX, an AMPA/kainate-type glutamate receptor antagonist, completely abolished the remaining fast-rise, fast-decay PSCs that most likely corresponded to AMPA receptor-mediated PSCs (FIG. 5B and 5C). These data clearly indicate that the iGNs could form functional synaptic connections with other excitatory glutamatergic neurons.

Next, spontaneous activity-dependent Ca²⁺ transients was examined either in homogenous populations of iENs or in mixtures of 80% iENs and 20% iGNs. In the homogenous population of iENs, the addition of bicuculline did not change the network activity, as measured by the synchronization of individual Ca^{2t} transients (FIG. 5D, 5F) Intriguingly, in mixed populations of iENs and iGNs, the addition of bicuculline increased the synchronization of the activity of the overall network by increasing the frequency of bursts of Ca²⁺ transients with a higher degree of synchronization (FIG. 5E, 5F) indicating that bicuculline removed the inhibitory effect of the iGNs within the neuronal network. This result is consistent with previous studies of cultured rodent neurons. These Ca²⁺-imaging data demonstrate the potential use of iGNs, together with iENs, to monitor the spontaneous network activity of iGNs or iENs within large populations of cells during drug screens.

Moreover, whether the iGNs could be used for interneuron-specific mechanistic studies was tested. Overexpression of MDGA1 (MAM-domain-containing glycosylphosphatidylinositol anchors 1) has been linked to autism and schizophrenia, in cortical neurons reduced inhibitory synapse numbers. However, whether the overexpression of MDGA1 in excitatory neurons or inhibitory neurons resulted in the reduced the inhibitory synaptic input remained unclear. To this end, MDGA1 was expressed in a homogenous population of iGNs via lentiviral transduction and measured inhibitory synapse density based on the number of VGAT-positive clusters. It was observed that MDGA1 overexpression significantly reduced inhibitory synapse density (FIG. 5G and 5H). Furthermore, compared with control cells, the iGNs that expressed MDGA1 displayed a significant reduction in sIPSC frequency (but not amplitude) (Control: 1.71±0.29 Hz, MDGA1: 0.92±0.25 Hz, p<0.05; Control: 20.1±1.58 pA, MDGA1: 16.66±1.38 pA) (FIG. 5I-5K). However, the expression of MDGA1 in iENs did not affect excitatory synaptic transmission, as assessed by sEPSCs (FIG. 12). Together, these results indicate that MDGA1 overexpression specifically and autonomously affected interneuron synapses; thus, the iGNs from this study may enable cell type-specific mechanistic studies of genes.

In the present study, a single-step, efficient, and reproducible method of generating a nearly pure population of human forebrain GABAergic neuronal cells (iGNs) is described based on the overexpression of selected TFs and microRNAs. Starting from hPSCs, we provided evidence that ASCL1, a proneural bHLH factor that is broadly expressed in the ventral brain, can induce a small fraction of MAP2-expressing neuronal cells within 7-10 days, consistent with previous reports (FIG. 1C). Co-expression of other factors, notably DLX2 and LHX6, together with A^{SA} significantly enhanced neuronal transformation while biasing the resultant neurons to differentiate almost exclusively into GABAergic interneurons. The results support a proneural activity of ASCL1 and indicate that other factors such as DLX2 and LHX6 confer hPSC-derived neurons with a GABAergic fate when they are expressed in conjunction with ASCL1. miR-9/9*-124 synergized with these TFs to more efficiently generate GABAergic neurons from either hESCs or hiPSCs (FIG. 1E). In support of these observations, GABAergic neurons could be reliably generated from multiple types of hPSCs, and mature neuronal properties in iGNs that were converted from additional hPSC lines were recorded (FIG. 8A-D). Although the iGNs appeared to uniformly express general forebrain GABAergic neuronal markers, multiple subtypes were found that expressed SST, CR, CB or NPY (FIG. 2E and 2G). Surprisingly, PV-positive neurons, a therapeutically important interneuron subtype that also originate from the MGE, were absent in the iGNs.

Compared to previous attempts to derive GABAergic neurons from non-neuronal human cells, the present method offers several advantages. First, the genetic gain-of-function approach bypasses the neural progenitor stage, thereby eliminating the need for various patterning factors and recombinant proteins (saving on costs and reducing experimental variability). Second, the protocol generates functional iGNs within a significantly shorter period of time (6-8 weeks, compared to 10-30 weeks), which enables more rapid turnaround of experiments. Third, the method primarily generates GABAergic neurons; few cells of other lineages are produced. These salient features enable a unique opportunity for in vitro assembly of microcircuits with neurons of defined identities and densities.

Indeed, distinct patterns of spontaneous neuronal network activity in dishes that contained discrete percentages of human excitatory (iENs) or inhibitory neurons (iGNs) were observed, and drug-induced alterations of the activity of the networks en bloc using Ca²⁺ imaging (FIG. 5D-F) were recorded. These proof-of-principle experiments clearly demonstrated the feasibility of the utilization of such a system to interrogate the formation of more complex circuits and network behaviors.

More importantly, the single-step nature of the present method permitted the generation of generate dox-inducible iGN (as well as iEN) hESC lines that can be synchronously differentiated into GABAergic neurons upon the addition of dox (FIG. 13). This single-step inducible system, in contrast to conventional multi-stage differentiation protocols, can produce large quantities of homogeneous induced neurons within a shorter period of time, which makes it an ideal platform for high-throughput screenings. Finally, this method enables neuronal subtype-specific characterization of the function of specific genes for disease modeling. For example, it was found that the overexpression of MDGA1, a schizophrenia and bipolar disorder susceptibility gene, in iGNs specifically reduced inhibitory synaptic transmission without altering excitatory synaptic transmission (FIG. 5G-K, and FIG. 12). Previously, this cell type-specific phenotyping was only achievable via the use of the Cre-lox system, which requires the generation of specific Cre driver lines and floxed alleles or by the generation of fluorescence-based reporter cell lines and subsequent fluorescence activated cell sorting (FACS)-mediated enrichment of specific cell types. This method therefore enables mechanistic and translation studies.

1-64. (canceled)

65. A method of directly converting a stem cell into a lineage specific cell comprising;
a) transfecting said stem cell with at least one expression vector comprising i) one or more cell lineage reprogramming factors operably linked to an inducible promoter and ii) a selection marker; and
b) inducing said transfected stem cell from operation a) with an inducing agent to directly convert said stem cell into a lineage specific cell.

66. The method of claim 65, wherein said at least one expression vector comprises a selection marker operably linked to a constitutive promoter; optionally
further comprising the operation of selecting the transfected stem cell for expression of the selection marker, prior to inducing the cells; optionally
wherein the selection marker is an antibiotic resistance gene selected from the group consisting of puromycin, blasticidin, hygromycin, zeocin and neomycin; optionally
wherein the constitutive promoter is selected from the group consisting of phosphoglycerate kinase (PGK), elongation factor 1-α (EF1α), β-actin and cytomegalovirus (CMV) enhancer/chicken β-actin promoter (CAG) and ubiquitin C (UBC).

67. The method of claim 65, further comprising the operation of transfecting the stem cell with an expression vector comprising a transactivator capable of inducing the inducible promoter in the presence of an inducer; optionally
wherein the inducer is selected from the group consisting of doxycycline and curate; optionally
wherein the expression vector is an integrating or non-integrating vector; optionally
wherein the integrating vector is a retroviral or lentiviral expression vector; optionally
wherein the non-integrating vector is a sendai virus, adeno-associated virus (AAV) or episomal DNA.

68. The method of claim 65 further comprising the operation of enriching the selected cells using one or more selection operations; optionally
wherein the selection operation is selected from the group consisting of antibiotic selection, fluorescence activated cell sorting (FACS), magnetic activated cell sorting (MACS), or single done isolation and expansion.

69. The method of claim 65, wherein the stem cell is an embryonic stem cell (ESC) or induced pluripotent stem cell (iPSC); optionally
wherein the stem cell is a primate or non-primate stem cell; optionally
wherein the primate stem cell is a human stem cell; optionally
wherein the stem cell is a stem cell line; optionally
wherein the stem cell line is cultured as a two-dimensional cell culture or a three-dimensional cell culture.

70. The method of claim 65, wherein the lineage specific cell is generated at an efficiency of at least 70%; optionally
wherein the lineage specific cell is a population of cells cultured as a two-dimensional cell culture or a three-dimensional cell culture; optionally
wherein the lineage specific cell is a cell of the ectoderm, mesoderm or endoderm lineage; optionally
wherein the cell of the ectoderm lineage is a neural cell; optionally
wherein the neural cell is selected from the group consisting of excitatory neurons, inhibitory neurons, dopamine neurons, serotonin neurons, medium spiny neurons, basal forebrain cholinergic neuron, oligodendrocytes, astrocytes and motor neurons; optionally
wherein the neural cell is an excitatory neuron; optionally
wherein the neural cell is an inhibitory neuron; optionally
wherein the neural cell is at least one cell of a cortical network; optionally
wherein the cell of the mesoderm lineage is a cardiac cell; optionally
wherein the cardiac cell is selected from the group consisting of cardiomyocytes, endothelial cells, vascular smooth muscle cells (VSMCs) and cardiac fibroblasts; optionally
wherein the cell of the endoderm lineage is a hepatic cell; optionally
wherein the hepatic cell is selected from the group consisting of hepatocytes, Kupffer cells stellate cells and sinusoidal endothelial cells; optionally
wherein the lineage specific cell is present in a homogenous population of cells.

71. The method of claim 65, wherein the one or more reprogramming factors is selected from the group consisting of a transcription factor, a chromatin remodeler, an epigenetic modifier and/or a non-coding RNA; optionally
wherein the non-coding RNA is microRNA; optionally
wherein the transcription factor is a neural transcription factor; optionally
wherein the neural transcription factor is one or more transcription factors selected from the group consisting of Ngn1, Ngn2, Ngn3, Neuro D1, Neuro D2, Brn1m Brn2m Brn3A, Brn3B, Brn3C, Brn4, Dlx1, Dlx2, Ascl1, phospho-dead mutant of the transcription factor Ascl1 (SA/SV-Ascl1), CTIP2, MYT1L, Olig1, Zic1 Nkx2.1, nkx2.2, Lhx2, Lhx3, Lhx6, Lhx8, SATB1, SATB2, Dlx5, Dlx6, Fezf2, Fev, Lmx1b, Lmx1a, Pitx3, Nurr1, FoxA2, Sox11, Atoh7, Olig2, Ptf1a, MEF2c, p55DD (dominant negative), Nkx6.1, Nkx6.2, Sox10, ST18, Myrf, Myt1, Zfp536, hes1, hes5, hes6, SOX2, SOX9, PAX5, NFIA, NFIB, NFIX, NICD, Islet1, Islet2, Irx3, Dbx2 and TAL1; optionally
wherein the transcription factor is a cardiac transcription factor; optionally
wherein the cardiac transcription factor is one or more transcription factors selected from the group consisting of Isl1, Mef2, Gata4, Tbx5, Nppa, Cx40, MESP1, MYOCD and ZFPM2, Baf60c, Hand2, Hopx, Hrt2, Pitx2c and nkx2.5; optionally
wherein the transcription factor is a hepatic transcription factor; optionally
wherein the hepatic transcription factor is one or more transcription factors selected from the group consisting of Hnf-1a, Hnf-1β, Hnf-3β, Hnf-3γ, Dbp, Hnf-4, Lrh-1, Fxrα, C/Ebpβ, Pxr, FOXA1, FOXA2, PROX1, HNF6, GATA6, PPARA, ZHX2, ONECUT2, ATF5, USF2, USF1, ZGPAT and NFIA; optionally
wherein the microRNA is microRNA-9/9* and/or microRNA-124, miRNA-219, miRNA-338, miRNA-1, miRNA-133 and miRNA187.

72. The method of claim 65, further comprising the operation of contacting the population of non-lineage specific cells with an expression vector comprising a fluorescent indicator; optionally
wherein the fluorescent indicator is a calcium indicator; optionaly
wherein the calcium indicator is GCaMP6.

73. A method of generating a directly convertible stem cell, said method comprising the operations of:
a) transfecting a stem cell with an expression vector comprising i) one or more cell lineage reprogramming factors operably linked to an inducible promoter and ii) a selection marker operably linked to a constitutive promoter; and
b) screening the transfected stem cell for expression of the selection marker to generate said directly convertible stem cell,
wherein said directly convertible stem cell is capable of direct conversion into an induced lineage specific cell,

74. A directly convertible stem cell comprising i) one or more reprogramming factors operably linked to an inducible promoter and ii) a selection marker operably linked to a constitutive promoter, wherein said directly convertible stem cell is capable of direct conversion into an induced lineage specific cell; optionally
wherein the directly convertible stem cell is a cell line.

75. A method of screening one or more factors and/or one or more genetic mutations that modulate a pre-selected activity of the induced lineage specific cell according to any one of the preceding claims, comprising the operations of:
a) culturing said induced lineage specific cell in the presence of one or more factors and/or one or more genetic mutations;
b) measuring the pre-selected activity of the lineage specific cell of operation a); and
c) comparing the measurement of b) relative to the measurement of the pre-selected activity in the lineage specific cell that has not been cultured in the presence of the said one or more factors or genetic mutations,
wherein the difference in the measurement of the pre-selected activity of the lineage specific cell in c) indicates that the one or more factors or genetic mutations modulates the pre-selected activity of the lineage specific cell.

76. The method of claim 75, wherein the one or more factors is selected from the group consisting of a drug, a growth factor, a small molecule, a biologic, a toxin, a stressor or a cell,

77. The method of claim 75, wherein the one or more genetic mutations is an engineered mutation or a naturally occurring mutation; optionally
wherein the engineered mutation is selected from the group consisting of site-directed mutation, deletion, duplication, inversion, copy-number variation, imprinting and random mutation; optionally
wherein the naturally occurring mutation is a polymorphism selected from the group consisting of single nucleotide polymorphism (SNP), microsatellite variation, small-scale insertion/deletion and polymorphic repetitive element,

78. The method of claim 75, wherein the pre-selected activity of the lineage specific cell is genetic activity or susceptibility to a disorder; optionally
wherein susceptibility to a disorder is determined by one or more assays selected from the group consisting of Ca2+ imaging, cell survival, intrinsic firing properties, measurement of Na+ channels, measurement of Ca2+ channels, measurement of K+ channels, synaptic activity, dendritic arborisation, axonal growth and targeting, neurotransmitter release and uptake, intracellular Ca2+ activity and extracellular activity; optionally
wherein the genetic activity is selected from the group consisting of gain-of-gene-function, loss-of-gene-function, gene knockdown, gene knockout and gene activation; optionally
wherein the genetic activity is achieved by small hairpin RNA (shRNA), small interfering RNA (siRNA) or CISPR-associated (Cas) endonuclease; optionally
wherein the disorder is a neural disorder; optionally
wherein the neural disorder is selected from the group consisting of schizophrenia, autism, Alzheimer's disease, Parkinson's, Depression, ADHD, dementia, epilepsy, Huntington's, Angelman syndrome, motor neuron disease (MND) and Dravet syndrome.

79. A kit for generating an induced lineage specific cell, comprising,
a) a directly convertible stem cell comprising i) one or more reprogramming factors operably linked to an inducible promoter and ii) a selection marker operably linked to a constitutive promoter, wherein said directly convertible stem cell is capable of direct conversion into an induced lineage specific cell; optionally
wherein the directly convertible stem cell is a cell line;
b) an inducer; and
optionally instructions for use.

80. A method of directly converting a stem cell into a lineage specific cell comprising:
a) transfecting said stem cell with at least one expression vector comprising i) one or more cell lineage reprogramming factors operably linked to a constitutive promoter; optionally
wherein the at least one expression vector further comprises a selection marker; optionally
wherein the selection marker is operably linked to a constitutive promoter; or
b) transfecting said stem cell with at least one expression vector comprising one or more cell lineage reprogramming factors operably linked to an inducible promoter.

81. A stem cell directly convertible into:
a) an inhibitory neuron comprising:
i) SA-ASCL1, DLX2, LHX6 and miR-9/9*-124 linked to a doxycycline inducible promoter; and
ii) a selection marker operably linked to a constitutive promoter; or
b) an excitatory neuron comprising:
i) NeuroD2 linked to a doxycycline inducible promoter; and
ii) a selection marker operably linked to a constitutive promoter.

82. A method of screening an agent using a cell obtained by a method of directly converting a stem cell into a lineage specific cell comprising:
a) transfecting said stem cell with at least one expression vector comprising i) one or more cell lineage reprogramming factors operably linked to an inducible promoter and ii) a selection marker; and
b) inducing said transfected stem cell from operation a) with an inducing agent to directly convert said stem cell into a lineage specific cell;
wherein the method of screening comprises:
i) contacting said cell with the agent;
ii) measuring a pre-selected activity of the agent on the cell and comparing this to a cell that has not been contacted with the agent; and
iii) detecting the activity of the agent on said cell.

83. A method of screening an agent using a cell obtained by a method of generating a directly convertible stem cell, said method comprising the operations of:
a) transfecting a stem cell with an expression vector comprising i) one or more cell lineage reprogramming factors operably linked to an inducible promoter and ii) a selection marker operably linked to a constitutive promoter; and
b) screening the transfected stem cell for expression of the selection marker to generate said directly convertible stem cell,
wherein said directly convertible stem cell is capable of direct conversion into an induced lineage specific cell;
wherein the method of screening comprises:
i) contacting said cell with the agent;
ii) measuring a pre-selected activity of the agent on the cell and comparing this to a cell that has not been contacted with the agent; and
iii) detecting the activity of the agent on said cell.

84. A method of screening an agent using a cell obtained by a method of directly converting a stem cell into a lineage specific cell comprising:
a) transfecting said stem cell with at least one expression vector comprising i) one or more cell lineage reprogramming factors operably linked to a constitutive promoter; optionally
wherein the at least one expression vector further comprises a selection marker;
optionally
wherein the selection marker is operably linked to a constitutive promoter; or
b) transfecting said stem cell with at least one expression vector comprising one or more cell lineage reprogramming factors operably linked to an inducible promoter;
wherein the method of screening comprises:
i) contacting said cell with the agent;
ii) measuring a pre-selected activity of the agent on the cell and comparing this to a cell that has not been contacted with the agent; and
iii) detecting the activity of the agent on said cell.


SEQUENCE LISTING (90 sequences)

SEQ ID NO: 1 - DNA, 22 bp, Artificial Sequence
  ANK2 Forward Primer
tcacaactgagtcatccatcac

SEQ ID NO: 2 - DNA, 21 bp, Artificial Sequence
  ANK Reverse Primer
attacctgcgatacagcttgg

SEQ ID NO: 3 - DNA, 21 bp, Artificial Sequence
  COUPTF1 Forward Primer
ctcaagaagtgcctcaaagtg

SEQ ID NO: 4 - DNA, 20 bp, Artificial Sequence
  COUPTF1 Reverse Primer
agatgtagccggacaggtag

SEQ ID NO: 5 - DNA, 22 bp, Artificial Sequence
  DARPP-32 Forward Primer
tctcaagtcgaagagacccaac

SEQ ID NO: 6 - DNA, 19 bp, Artificial Sequence
  DARPP-32 Reverse Primer
tgcaggtgagactcagcaa

SEQ ID NO: 7 - DNA, 22 bp, Artificial Sequence
  DAT Forward Primer
agagacgaagaccccaggaagt

SEQ ID NO: 8 - DNA, 25 bp, Artificial Sequence
  DAT Reverse Primer
gaggttgaagagtagaagttgccct

SEQ ID NO: 9 - DNA, 20 bp, Artificial Sequence
  DLX1 Forward Primer
ggctgtttgccaattcaggg

SEQ ID NO: 10 - DNA, 20 bp, Artificial Sequence
  DLX1 Revese Primer
ctccccgtgcgcttaaagta

SEQ ID NO: 11 - DNA, 22 bp, Artificial Sequence
  DLX5 Forward Primer
acagagacttcacgactcccag

SEQ ID NO: 12 - DNA, 20 bp, Artificial Sequence
  DLX5 Reverse Primer
tgtggggctgctctggtcta

SEQ ID NO: 13 - DNA, 25 bp, Artificial Sequence
  DLX6 Forward Primer
tggtgaaagagaagcattttggact

SEQ ID NO: 14 - DNA, 24 bp, Artificial Sequence
  DLX6 Reverse Primer
agagaagggctgttatgtgaggaa

SEQ ID NO: 15 - DNA, 20 bp, Artificial Sequence
  EN1 Forward Primer
tctcgctgtctctccctctc

SEQ ID NO: 16 - DNA, 20 bp, Artificial Sequence
  EN1 Reverse Primer
cgtggcttactccccattta

SEQ ID NO: 17 - DNA, 19 bp, Artificial Sequence
  ERBB4 Forward Primer
tcgtgtcgccgcttcagta

SEQ ID NO: 18 - DNA, 23 bp, Artificial Sequence
  ERBB4 Reverse Primer
gagccattctcaaactcccgaaa

SEQ ID NO: 19 - DNA, 21 bp, Artificial Sequence
  FOXG1 Forward Primer
agccgccagatttccatgtgt

SEQ ID NO: 20 - DNA, 20 bp, Artificial Sequence
  FOXG1 Reverse Primer
ctcaaggtctgcgtccacca

SEQ ID NO: 21 - DNA, 24 bp, Artificial Sequence
  GAD1 Forward Primer
caaacatttatcaacatgcgcttc

SEQ ID NO: 22 - DNA, 21 bp, Artificial Sequence
  GAD1 Reverse Primer
ctatgacactggagacaaggc

SEQ ID NO: 23 - DNA, 21 bp, Artificial Sequence
  GAD2 Forward Primer
atcctggttgactgcagagac

SEQ ID NO: 24 - DNA, 20 bp, Artificial Sequence
  GAD2 Reverse Primer
ccagtggagagctggttgaa

SEQ ID NO: 25 - DNA, 20 bp, Artificial Sequence
  GAPDH Forward Primer
ctctgctcctcctgttcgac

SEQ ID NO: 26 - DNA, 20 bp, Artificial Sequence
  GAPDH Reverse Primer
gcgcccaatacgaccaaatc

SEQ ID NO: 27 - DNA, 20 bp, Artificial Sequence
  GFAP Forward Primer
cggatcaccattcccgtgca

SEQ ID NO: 28 - DNA, 22 bp, Artificial Sequence
  GFAP Reverse Primer
ttgaggtggccttctgacacag

SEQ ID NO: 29 - DNA, 21 bp, Artificial Sequence
  GPHN Forward Primer
caccagaattcgcacttcaag

SEQ ID NO: 30 - DNA, 21 bp, Artificial Sequence
  GPHN Reverse Primer
agacagtaatgccaggacaag

SEQ ID NO: 31 - DNA, 21 bp, Artificial Sequence
  GRIA1 Foward Primer
cttcccggaccaaagtgatag

SEQ ID NO: 32 - DNA, 20 bp, Artificial Sequence
  GRIA1 Reverse Primer
tgatggaaaatacggagccc

SEQ ID NO: 33 - DNA, 25 bp, Artificial Sequence
  GRIA2 Forward Primer
tggagtgaagtggacaaaatggttg

SEQ ID NO: 34 - DNA, 25 bp, Artificial Sequence
  GRIA2 Reverse Primer
gtcttattctcaagcccagaggtgt

SEQ ID NO: 35 - DNA, 20 bp, Artificial Sequence
  GRIN1 Forward Primer
aggaacccctcggacaagtt

SEQ ID NO: 36 - DNA, 19 bp, Artificial Sequence
  GRIN1 Reverse Primer
ccgcactctcgtagttgtg

SEQ ID NO: 37 - DNA, 19 bp, Artificial Sequence
  GRIN2A Forward Primer
gggctgggacatgcagaat

SEQ ID NO: 38 - DNA, 22 bp, Artificial Sequence
  GRIN2A Reverse Primer
cgtctttggaacagtagagcaa

SEQ ID NO: 39 - DNA, 21 bp, Artificial Sequence
  GRIN2B Forward Primer
gtagccatgaatgagaccgac

SEQ ID NO: 40 - DNA, 20 bp, Artificial Sequence
  GRIN2B Reverse Primer
ggatcggggtgagagtctgt

SEQ ID NO: 41 - DNA, 20 bp, Artificial Sequence
  GRP Forward Primer
accgtgctgaccaagatgta

SEQ ID NO: 42 - DNA, 22 bp, Artificial Sequence
  GRP Reverse Primer
gctccctctctcagaaacagaa

SEQ ID NO: 43 - DNA, 21 bp, Artificial Sequence
  HPRT1 Forward Primer
gctttccttggtcaggcagta

SEQ ID NO: 44 - DNA, 21 bp, Artificial Sequence
  HPRT1 Reverse Primer
acttcgtggggtccttttcac

SEQ ID NO: 45 - DNA, 19 bp, Artificial Sequence
  MAP2 Forward Primer
caacggagagctgacctca

SEQ ID NO: 46 - DNA, 21 bp, Artificial Sequence
  MAP2 Revese Primer
ctacagcctcagcagtgacta

SEQ ID NO: 47 - DNA, 23 bp, Artificial Sequence
  MAPT Forward Primer
gaagattgggtccctggacaata

SEQ ID NO: 48 - DNA, 20 bp, Artificial Sequence
  MAPT Reverse Primer
aggtcagcttgtgggtttca

SEQ ID NO: 49 - DNA, 22 bp, Artificial Sequence
  NANOG Forward Primer
ggacactggctgaatccttcct

SEQ ID NO: 50 - DNA, 22 bp, Artificial Sequence
  NANOG Reverse Primer
ctccaaccatactccaccctcc

SEQ ID NO: 51 - DNA, 21 bp, Artificial Sequence
  NCAM Forward Primer
ggctccttggactcatctttc

SEQ ID NO: 52 - DNA, 21 bp, Artificial Sequence
  NCAM Reverse Primer
gacatcacctgctacttcctg

SEQ ID NO: 53 - DNA, 19 bp, Artificial Sequence
  NESTIN Forward Primer
ggctgagggacatcttgag

SEQ ID NO: 54 - DNA, 20 bp, Artificial Sequence
  NESTIN Reverse Primer
tgcgggctactgaaaagttc

SEQ ID NO: 55 - DNA, 20 bp, Artificial Sequence
  OCT4 Forward Primer
aggtgtcggaatgcgtggtg

SEQ ID NO: 56 - DNA, 20 bp, Artificial Sequence
  OCT4 Reverse Primer
aatccgggagcgggcttcta

SEQ ID NO: 57 - DNA, 21 bp, Artificial Sequence
  OLIG2 Forward Primer
ccccaccgactcatctttcct

SEQ ID NO: 58 - DNA, 23 bp, Artificial Sequence
  OLIG2 Reverse Primer
ccaacagaacccccaaataaccc

SEQ ID NO: 59 - DNA, 16 bp, Artificial Sequence
  PCP2 Forward Primer
cgggccagaccaccaa

SEQ ID NO: 60 - DNA, 20 bp, Artificial Sequence
  PCP2 Reverse Primer
tgtcacacgttggtcatcca

SEQ ID NO: 61 - DNA, 22 bp, Artificial Sequence
  PROX1 Forward Primer
cctcccattactcagacccgtg

SEQ ID NO: 62 - DNA, 21 bp, Artificial Sequence
  PROX1 Reverse Primer
aggctcccgcttagaaactgt

SEQ ID NO: 63 - DNA, 19 bp, Artificial Sequence
  PSD95 Forward Primer
agctggagcaggagttcac

SEQ ID NO: 64 - DNA, 20 bp, Artificial Sequence
  PSD95 Reverse Primer
acacgcttcaccttgtggta

SEQ ID NO: 65 - DNA, 23 bp, Artificial Sequence
  SATB1 Forward Primer
agagctgtcagtggaaggaaaca

SEQ ID NO: 66 - DNA, 24 bp, Artificial Sequence
  SATB1 Reverse Primer
gtggcactgttgaacgaaacaaat

SEQ ID NO: 67 - DNA, 21 bp, Artificial Sequence
  SCN1A Forward Primer
agagggcgagcaaaggatgtg

SEQ ID NO: 68 - DNA, 20 bp, Artificial Sequence
  SCN1A Reverse Primer
cgtcggggcacaaacaagga

SEQ ID NO: 69 - DNA, 20 bp, Artificial Sequence
  SERT Forward Primer
tgctggcttttgctagctac

SEQ ID NO: 70 - DNA, 20 bp, Artificial Sequence
  SERT Reverse Primer
gaagctcgtcatgcagttca

SEQ ID NO: 71 - DNA, 21 bp, Artificial Sequence
  SOX2 Forward Primer
atcccatccacactcacgcaa

SEQ ID NO: 72 - DNA, 21 bp, Artificial Sequence
  SOX2 Reverse Primer
accctccccaggttttctctg

SEQ ID NO: 73 - DNA, 25 bp, Artificial Sequence
  SOX6 Forward Primer
agttcctctggctgactctatctgt

SEQ ID NO: 74 - DNA, 21 bp, Artificial Sequence
  SOX6 Reverse Primer
aaacctcactgcttcccaccc

SEQ ID NO: 75 - DNA, 21 bp, Artificial Sequence
  SP8 Forward Primer
tgcttgctcccgaatcagacg

SEQ ID NO: 76 - DNA, 20 bp, Artificial Sequence
  SP8 Reverse Primer
tcttcctggcacccccaaca

SEQ ID NO: 77 - DNA, 20 bp, Artificial Sequence
  SYN1 Forward Primer
atgtcctggaagtcatgctg

SEQ ID NO: 78 - DNA, 22 bp, Artificial Sequence
  SYN1 Reverse Primer
ccccaatcacaaagaaatgctc

SEQ ID NO: 79 - DNA, 21 bp, Artificial Sequence
  TPH1 Forward Primer
cttgggagaattgggcaaaac

SEQ ID NO: 80 - DNA, 22 bp, Artificial Sequence
  TPH1 Reverse Primer
gagacacagttcagatcccttc

SEQ ID NO: 81 - DNA, 21 bp, Artificial Sequence
  TPH2 Forward Primer
atggctcagatcccctctaca

SEQ ID NO: 82 - DNA, 21 bp, Artificial Sequence
  TPH2 Reverse Primer
ggatccgcaagtagtggaaca

SEQ ID NO: 83 - DNA, 22 bp, Artificial Sequence
  VGAT Forward Primer
agatgatgagaaacaaccccag

SEQ ID NO: 84 - DNA, 20 bp, Artificial Sequence
  VGAT Reverse Primer
cacgacaagcccaaaatcac

SEQ ID NO: 85 - DNA, 20 bp, Artificial Sequence
  VGLUT1 Forward Primer
tcaagtccccgattccgtgc

SEQ ID NO: 86 - DNA, 22 bp, Artificial Sequence
  VGLUT1 Reverse Primer
tgcgattttggttgtttcccca

SEQ ID NO: 87 - DNA, 20 bp, Artificial Sequence
  VGLUT2 Forward Primer
tggggctacatcatcactca

SEQ ID NO: 88 - DNA, 20 bp, Artificial Sequence
  VGLUT2 Reverse Primer
gaagtatggcagctccgaaa

SEQ ID NO: 89 - DNA, 23 bp, Artificial Sequence
  ZEB2 Forward Primer
gggcatatctgtgtcccaatcca

SEQ ID NO: 90 - DNA, 21 bp, Artificial Sequence
  ZEB2 Reverse Primer
caccctcctctacttcgtgca